(19) United States
(12) Patent Application Publication
(10) Pub. No.: US 2021/0404002 A1
(43) Pub. Date: Dec. 30, 2021

(54) Quantitative Algorithm for Endometriosis

(71) Applicant: YALE UNIVERSITY, New Haven, CT (US)

(72) Inventor: Hugh Taylor, Easton, CT (US)

(21) Appl. No.: 17/289,882

(22) PCT Filed: Oct. 31, 2019
(86) PCT No.: PCT/US19/59006

§ 371 Date: Apr. 29, 2021

Related U.S. Application Data
(60) Provisional application No. 62/753,265, filed on Oct. 31, 2018.

(51) Int. Cl. C12Q 1/6883; G16B 20/00; G16B 40/20

(52) U.S. Cl. CPC C12Q 1/6883; G16B 20/00; C12Q 2600/178; C12Q 2600/158; G16B 40/20

(57) ABSTRACT

Disclosed herein are methods for developing and using quantitative algorithms, cutoff points, and numerical scores based upon the expression level of at least one miRNA that is associated with endometriosis.

### CROSS-REFERENCE TO RELATED APPLICATIONS

This application claims the benefit of U.S. Provisional Application No. 62/753,265, filed on Oct. 31, 2018, which application is incorporated herein by reference in its entirety.

### STATEMENT REGARDING FEDERALLY SPONSORED RESEARCH OR DEVELOPMENT

This invention was made with government support under HD 076422 awarded by the National Institutes of Health (NIH). The government has certain rights in the invention.

### BACKGROUND

Endometriosis is traditionally a challenging diagnosis with a significant (multiple-year) delay between onset of symptoms and clinically verified diagnosis. This may be due to a combination of factors: the fact that common symptoms of endometriosis (e.g., pelvic pain, dysmenorrhea) can be interpreted as merely extreme variants of menstruation, and that a the current “gold standard” diagnosis of endometriosis is via laparoscopy (see, for e.g. Ballard et al. Fertil Steril. 2006 November; 86(5):1296-301.) Even in the event of successful diagnosis by laparoscopy, the invasiveness of laparoscopy and post-operative pain associated with it makes it unsuitable for ongoing repeat monitoring of the disease in case of relapse or unsuccessful initial treatment.

MicroRNAs (miRNAs) are a class of highly conserved small endogenous non-coding, functional RNA molecules of 19-24 nucleotides; they may control the translation and stability of targeted RNAs by base-pairing to complementary sites and induce repression or degradation of messenger RNA transcripts. miRNAs have been associated with a wide array of disease processes, making miRNAs an interesting avenue of exploration as biomarkers for diseases such as endometriosis.

### SUMMARY OF THE INVENTION

In some aspects, the present disclosure provides for a method of assessing endometriosis, comprising: (a) inputting a level of at least one miRNA into an algorithm, wherein the at least one miRNA is selected from the group consisting of miR-18, miR-18a, miR-18a-5p, miR-34c, miR-125, miR-125b, miR-125b-5p, miR-126, miR-135, miR-141, miR-145, miR-143, miR-143-3p, miR-145-5p, miR-150, miR-150-5p, miR-200, miR-200a, miR-200b, miR-214, miR-342, miR-342-3p, miR-449, miR-449a, miR-451, miR-451a, miR-500, miR-500a, miR-500a-3p, miR-553, miR-3613, miR-3613-5p, miR-4668, miR-6755, miR-6755-3p, and let-7b, (b) quantitatively assessing the performance of the at least one miRNA or the algorithm using receiver operating characteristic (ROC) analysis and understanding AUC (Area Under the Curve), (c) combining at least one of the at least one miRNA into a mathematical formula, (d) assigning weights to at least one of the at least one miRNA within the formula, and (e) using the assigned weights to develop a quantitative algorithm to distinguish the presence or absence of disease. In some embodiments, the method further comprises one or more of: (f) developing a quantitative algorithm to determine the extent, severity, or stage of disease, (g) developing a quantitative algorithm to determine a treatment approach (e.g., oral contraceptives, disease-specific therapy, surgical intervention), (h) developing a quantitative algorithm to select the appropriate dose for a medical treatment, (i) developing a quantitative algorithm to determine whether a patient is likely to respond to a particular medical or surgical treatment, (j) developing a quantitative algorithm to monitor response to treatment, and (k) developing a quantitative algorithm to monitor disease progression. In some embodiments, the method further comprises deriving a numerical value or score from the quantitative algorithm or mathematical formula using at least one miRNA. In some embodiments, the method further comprises establishing cutoff values for the derived numerical value or score from the quantitative algorithm for endometriosis. In some embodiments, the method comprises at least one of: (a) establishing cutoff values for at least one miRNA, (b) establishing cutoff values for the derived numerical value or score from one or more miRNAs in order distinguish the presence or absence of disease, (c) establishing cutoff values for the derived numerical value or score from one or more miRNAs in order determine the extent, severity, or stage of disease, (d) establishing cutoff values for the derived numerical value or score from one or more miRNAs in order to determine the right treatment approach (e.g., oral contraceptives, disease-specific therapy, surgical intervention), (e) establishing cutoff values for the derived numerical value or score from one or more miRNAs in order to select the appropriate dose for a medical treatment, (f) establishing cutoff values for the derived numerical value or score from one or more miRNAs in order to determine whether a patient is likely to respond to a particular medical or surgical treatment, (g) establishing cutoff values for the derived numerical value or score from one or more miRNAs in order to monitor response to treatment, and (h) establishing cutoff values for the derived numerical value or score from one or more miRNAs in order to monitor disease progression. In some embodiments, the method further comprises obtaining results derived from a quantitative algorithm for endometriosis. In some embodiments, the quantitative algorithm is for determining the treatment approach (e.g., oral contraceptives, disease-specific therapy, surgical intervention). In some embodiments, the quantitative algorithm is for selecting the appropriate dose for a medical treatment. In some embodiments, the quantitative algorithm is for determining whether a patient is likely to respond to a particular medical or surgical treatment. In some embodiments, the quantitative algorithm is used for monitoring response to treatment. In some embodiments, the quantitative algorithm is used for monitoring disease progression. In some embodiments, the at least one miRNA is selected from the group consisting of miR-135, miR-449a, miR-34c, miR-200a, miR-200b, miR-141, miR-125b-5p, miR-150-5p, miR-143-3p, miR-500a-3p, miR-18a-5p, miR-6755-3p, and miR-3613-5p. In some embodiments, the at least one miRNA comprises: (i) miR-150, miR-342, miR-451, and let-7b; (ii) miR-125, miR-451, and miR-3613; (iii) miR-125, miR-150, miR-342, and miR-451; (iv) miR-125, miR-150, miR-342, miR-451, let-7b, and miR-3613; or (v) miR-125 and miR-342. In some embodiments, the quantitative algorithm is a fisher discriminant algorithm or a support vector machine algorithm.

In some aspects, the present disclosure provides for a method of diagnosing a subject suspected of having endometriosis comprising: (a) obtaining a blood, serum, plasma, saliva, sputum, urine, lymphatic fluid, synovial fluid, cerebrospinal fluid, stool, or mucus sample from the subject, wherein the blood, serum, plasma, saliva, sputum, urine, lymphatic fluid, synovial fluid, cerebrospinal fluid, stool, or mucus sample comprises miRNA associated with endometriosis; (b) detecting a level of at least one miRNA selected from the group consisting of miR-125, miR-150, miR-342, miR-145, miR-143, miR-500, miR-451, miR-18, miR-214, miR-126, miR-6755, miR-3613, miR-553, miR-4668, let-7b, miR-135, miR-449a, miR-34c, miR-200a, miR-200b, miR-141, miR-125b-5p, miR-150-5p, miR-342-3p, miR-145-5p, miR-143-3p, miR-500a-3p, miR-451a, miR-18a-5p, miR-6755-3p, and miR-3613-5p; (c) comparing the level of the at least one miRNA with a cutoff value for the at least one miRNA derived from a quantitative algorithm for endometriosis; and (d) diagnosing the subject with endometriosis based on the comparison of the level of the at least one miRNA to the cutoff value. In some embodiments, the at least one miRNA is the combination of miR-150, miR-342, miR-451 and let-7b. In some embodiments, the at least one miRNA is the combination of an increased level of miR-150, an increased level of miR-342, an increased level of miR-451 and a decreased level of let-7b relative to the level in a comparator control. In some embodiments, the increased level of miR-150 is at least 9-fold increased relative to the level in the comparator control; the increased level of miR-451 is at least 2-fold increased relative to the level in the comparator control; the decreased level of let-7b is at least 8-fold decreased relative to the level in the comparator control, or any combination thereof. In some embodiments, the comparator control is the level of the miRNA in a population without endometriosis. In some embodiments, the at least one miRNA is the combination of miR-125, miR-451, and miR-3613. In some embodiments, the at least one miRNA is the combination of an increased level of miR-125, an increased level of miR-451, and a decreased level of miR-3613 relative to the level in a comparator control. In some embodiments, the increased level of miR-125 is at least five-fold increased relative to the level in the comparator control; the increased level of miR-451 is at least 2-fold increased relative to the level in the comparator control; the decreased level of miR-3613 is at least four-fold decreased relative to the level in the comparator control, or any combination thereof. In some embodiments, the comparator control is the level of the miRNA in a population without endometriosis. In some embodiments, the method further comprises administering a treatment to the subject for endometriosis. In some embodiments, the treatment is at least one treatment selected from the group consisting of hormone therapy, chemotherapy, immunotherapy, and surgical treatment.

In some aspects, the present disclosure provides for a method of detecting endometriosis using a quantitative polymerase chain reaction (qPCR) machine or sequencing machine comprising: (a) introducing nucleic acids into the qPCR machine or sequencing machine, wherein nucleic acids are derived from a sample obtained from a female subject with endometriosis or with symptoms of endometriosis; (b) using the qPCR machine or sequencing machine to detect a level of at least one miRNA in the nucleic acids, wherein the at least one miRNA is selected from the group consisting of miR-18, miR-18a, miR-18a-5p, miR-34c, miR-125, miR-125b, miR-125b-5p, miR-126, miR-135, miR-141, miR-145, miR-143, miR-143-3p, miR-145-5p, miR-150, miR-150-5p, miR-200, miR-200a, miR-200b, miR-214, miR-342, miR-342-3p, miR-449, miR-449a, miR-451, miR-451a, miR-500, miR-500a, miR-500a-3p, miR-553, miR-3613, miR-3613-5p, miR-4668, miR-6755, miR-6755-3p, and let-7b, thereby obtaining a detected level of the at least one miRNA; (c) introducing the detected level of the at least one miRNA into a trained algorithm wherein the algorithm: (i) is trained using data from endometriosis subjects; (ii) assigns weights to the at least one miRNA selected from the group consisting of miR-18, miR-18a, miR-18a-5p, miR-34c, miR-125, miR-125b, miR-125b-5p, miR-126, miR-135, miR-141, miR-145, miR-143, miR-143-3p, miR-145-5p, miR-150, miR-150-5p, miR-200, miR-200a, miR-200b, miR-214, miR-342, miR-342-3p, miR-449, miR-449a, miR-451, miR-451a, miR-500, miR-500a, miR-500a-3p, miR-553, miR-3613, miR-3613-5p, miR-4668, miR-6755, miR-6755-3p, and let-7b; and (iii) detects endometriosis based on the assigned weights of the at least one miRNA; and (d) using the trained algorithm to detect presence of absence of endometriosis in the female subject. In some embodiments, the at least one miRNA is selected from the group consisting of miR-135, miR-449a, miR-34c, miR-200a, miR-200b, miR-141, miR-125b-5p, miR-150-5p, miR-143-3p, miR-500a-3p, miR-18a-5p, miR-6755-3p, and miR-3613-5p. In some embodiments, the at least one miRNA comprises: (i) miR-150, miR-342, miR-451, and let-7b; (ii) miR-125, miR-451, and miR-3613; (iii) miR-125, miR-150, miR-342, and miR-451; (iv) miR-125, miR-150, miR-342, miR-451, let-7b, and miR-3613; or (v) miR-125 and miR-342. In some embodiments, the trained algorithm is a support vector machine algorithm or fisher discriminant algorithm. In some embodiments, the method uses a qPCR machine. In some embodiments, the method uses a sequencing machine. In some embodiments, the sequencing machine is a next-generation sequencing machine. In some embodiments, the method comprises using the trained algorithm to detect the presence of endometriosis in the female subject. In some embodiments, further comprises administering a treatment to the female subject for the endometriosis. In some embodiments, the treatment is selected from the group consisting of: hormone therapy, chemotherapy, immunotherapy, and surgical treatment. In some embodiments, the sample is a blood, plasma or serum sample. In some embodiments, the sample is a saliva or sputum sample. In some embodiments, the at least one miRNA is the combination of miR-150, miR-342, miR-451 and let-7b. In some embodiments, the endometriosis is detected when an increased level of miR-150, an increased level of miR-342, an increased level of miR-451 and a decreased level of let-7b relative to the level in a comparator control is detected. In some embodiments, the increased level of miR-150 is at least 9-fold increased relative to the level in the comparator control; the increased level of miR-451 is at least 2-fold increased relative to the level in the comparator control; the decreased level of let-7b is at least 8-fold decreased relative to the level in the comparator control, or any combination thereof. In some embodiments, the comparator control is the level of the miRNA in a population without endometriosis. In some embodiments, the at least one miRNA is the combination of miR-125, miR-451, and miR-3613. In some embodiments, the endometriosis is detected when an increased level of miR-125, an increased level of miR-451, and a decreased level of miR-3613 relative to the level in a comparator control is detected. In some embodiments, the increased level of miR-125 is at least five-fold increased relative to the level in the comparator control; the increased level of miR-451 is at least 2-fold increased relative to the level in the comparator control; the decreased level of miR-3613 is at least four-fold decreased relative to the level in the comparator control, or any combination thereof. In some embodiments, the comparator control is the level of the miRNA in a population without endometriosis. In some embodiments, the treatment is selected from the group consisting of oral contraceptive pill, GNRH agonists, aromatase inhibitors, and progesterone.

In some embodiments, the present disclosure provides a method of assessing endometriosis, comprising: utilizing a level of at least one miRNA in the algorithm, wherein the miRNA is selected from the group consisting of miR-125, miR-150, miR-342, miR-145, miR-143, miR-500, miR-451, miR-18, miR-214, miR-126, miR-6755, miR-3613, miR-553, miR-4668, let-7b, miR-135, miR-449a, miR-34c, miR-200a, miR-200b, miR-141, miR-125b-5p, miR-150-5p, miR-342-3p, miR-145-5p, miR-143-3p, miR-500a-3p, miR-451a, miR-18a-5p, miR-6755-3p, and miR-3613-5p; quantitatively assessing the performance of at least one miRNA or algorithm combining one or more miRNAs using receiver operating characteristic (ROC) analysis and understanding AUC (Area Under the Curve); combining at least one miRNA into a mathematical formula; assigning weights to at least one of the miRNAs within the formula; and developing a quantitative algorithm to distinguish the presence or absence of disease.

In some embodiments, the method further comprises developing a quantitative algorithm to determine the extent, severity, or stage of disease, developing a quantitative algorithm to determine the right treatment approach (e.g., oral contraceptives, disease-specific therapy, surgical intervention), developing a quantitative algorithm to select the appropriate dose for a medical treatment, developing a quantitative algorithm to determine whether a patient is likely to respond to a particular medical or surgical treatment, developing a quantitative algorithm to monitor response to treatment, developing a quantitative algorithm to monitor disease progression, or a combination thereof.

In some embodiments, the method further comprises deriving a numerical value or score from the quantitative algorithm or mathematical formula using at least one miRNA.

In some embodiments, the method further comprises establishing cutoff values for the derived numerical value or score from the quantitative algorithm for endometriosis. In some embodiments, the method comprises establishing cutoff values for at least one miRNA; establishing cutoff values for the derived numerical value or score from one or more miRNAs in order distinguish the presence or absence of disease; establishing cutoff values for the derived numerical value or score from one or more miRNAs in order determine the extent, severity, or stage of disease; establishing cutoff values for the derived numerical value or score from one or more miRNAs in order to determine the right treatment approach (e.g., oral contraceptives, disease-specific therapy, surgical intervention); establishing cutoff values for the derived numerical value or score from one or more miRNAs in order to select the appropriate dose for a medical treatment; establishing cutoff values for the derived numerical value or score from one or more miRNAs in order to determine whether a patient is likely to respond to a particular medical or surgical treatment; establishing cutoff values for the derived numerical value or score from one or more miRNAs in order to monitor response to treatment; establishing cutoff values for the derived numerical value or score from one or more miRNAs in order to monitor disease progression, or a combination thereof.

In some embodiments, the method further comprises the step of obtaining results derived from a quantitative algorithm for endometriosis. In some embodiments, the quantitative algorithm is for determining the treatment approach (e.g., oral contraceptives, disease-specific therapy, surgical intervention). In some embodiments, the quantitative algorithm is for selecting the appropriate dose for a medical treatment. In some embodiments, the quantitative algorithm is for determining whether a patient is likely to respond to a particular medical or surgical treatment. In some embodiments, the quantitative algorithm is used for monitoring response to treatment. In some embodiments, the quantitative algorithm is used for monitoring disease progression.

In some embodiments, the current disclosure provides a method of diagnosing a subject suspected of having endometriosis comprising: obtaining a saliva, sputum, urine, lymphatic fluid, synovial fluid, cerebrospinal fluid, stool, or mucus sample from the subject, wherein the saliva, sputum, urine, lymphatic fluid, synovial fluid, cerebrospinal fluid, stool, or mucus sample comprises miRNA associated with endometriosis; detecting a level of at least one miRNA selected from the group consisting of miR-125, miR-150, miR-342, miR-145, miR-143, miR-500, miR-451, miR-18, miR-214, miR-126, miR-6755, miR-3613, miR-553, miR-4668, let-7b, miR-135, miR-449a, miR-34c, miR-200a, miR-200b, miR-141, miR-125b-5p, miR-150-5p, miR-342-3p, miR-145-5p, miR-143-3p, miR-500a-3p, miR-451a, miR-18a-5p, miR-6755-3p, and miR-3613-5p; comparing the level of the at least one miRNA with a cutoff value for the at least one miRNA derived from a quantitative algorithm for endometriosis; and diagnosing the subject with endometriosis based on the comparison of the level of the at least one miRNA to the cutoff value.

In some embodiments, the method includes detecting the level of the combination of miR-150, miR-342, miR-451 and let-7b. In some embodiments, the method includes detecting a combination of an increased level of miR-150, an increased level of miR-342, an increased level of miR-451 and a decreased level of let-7b relative to the level in a comparator control. In some embodiments, the comparator control is the level of the miRNA in a population without endometriosis.

In some embodiments, the method includes detecting the level of the combination of miR-125, miR-451, and miR-3613. In some embodiments, the method includes detecting a combination of an increased level of miR-125, an increased level of miR-451, and a decreased level of miR-3613 relative to the level in a comparator control. In some embodiments, the comparator control is the level of the miRNA in a population without endometriosis.

In some embodiments, the method further comprises the step of treating the subject for endometriosis. In some embodiments, the treatment is hormone therapy, chemotherapy, immunotherapy, surgical treatment, or a combination thereof.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1 depicts the analytical process by which a quantitative algorithm is used to calculate a numerical value or score for single or multiple individuals. An example algorithm is shown, and the numerical value or score for multiple individuals is calculated based on the algorithm. The numerical values are graphed for multiple individuals in order to determine the overall performance of the algorithm. The values are illustratively show separately for patients who truly have endometriosis, or ‘True Endo Patients’, and those who truly do not have endometriosis, or ‘True Control Patients’. True values are based on assessment via laparoscopy. Such an analytical process can also be used to determine the right cutoff point for the algorithm in order to determine whether a patient does or does not have endometriosis based on the numerical value derived from the algorithm.

FIG. 2, comprising FIG. 2A and FIG. 2E, depicts the process by which a ROC curve is generated for an individual miRNA. FIG. 2A depicts different expression levels between endometriosis patients and controls, normalized relative to small nuclear RNA U6 levels, for illustrative microRNA. The ROC curve is calculated, along with the sensitivity and specificity values. FIG. 2B depicts a summary of ROC curve analysis for illustrative microRNA. FIG. 2C depicts a summary of sensitivity and specificity values, and confidence intervals for illustrative microRNA. FIG. 2D depicts the ROC curve values at each sensitivity and specificity value for illustrative microRNA. FIG. 2E depicts a graphical ROC curve for illustrative microRNA with area under the curve (AUC) of 0.7962.

FIG. 3 depicts an illustrative Areas Under the Curve (AUC) analysis in order to measure the performance of individual miRNAs. Receiver operating characteristic (ROC) analysis is also used to understand AUC for an algorithm using a combination of miRNAs. An understanding of the ROC analysis and AUC is used to determine the algorithm's sensitivity and specificity for the disease.

FIG. 4 depicts and illustrative report of the results to the patient, physician, or other healthcare provider or clinic. In the report, which can be transmitted digitally or by other methods, the values of individual miRNAs is shown. The assessment that the patient does not have endometriosis, in this case, is based on one or a combination of miRNAs that are combined using a quantitative algorithm. The numerical value derived from the algorithm is compared against a cutoff point in order to determine that this example patient does not have endometriosis.

FIG. 5 demonstrates the differential miRNA expression levels between controls and minimal/mild (Stage I+II) vs. moderate/severe (Stage III+IV) endometriosis. Cutoff values for miRNA levels that correlate with disease stage and severity would be determined from this analysis.

FIG. 6 depicts exemplary quantitative PCR (qt-PCR, or Q-PCR) analyses of the expression of a subset of miRNA in endometriosis and control subjects. A significant increase in expression of miR-125, miR-150, miR-342, and miR-451 was identified in patients with endometriosis, and significantly reduced expression of miR-3613 and let-7b.

FIG. 7 depicts exemplary qt-PCR analyses of the expression of a subset of miRNA in between endometriosis patients who received hormonal therapy and those that did not.

FIG. 8 depicts exemplary qt-PCR analyses of the expression of a subset of miRNA in endometriosis patients when the sample was collected either during the proliferative phase of the menstrual cycle or the secretory phase of the menstrual cycle.

FIG. 9 depicts exemplary receiver operating characteristic (ROC) curves and the areas under the ROC curve (AUC) were established to evaluate the diagnostic value of individual plasma microRNAs for differentiating between endometriosis and control groups.

FIG. 10 depicts exemplary receiver operating characteristic (ROC) curves and the areas under the ROC curve (AUC) for two subsets of miRNA which yielded high diagnostic value.

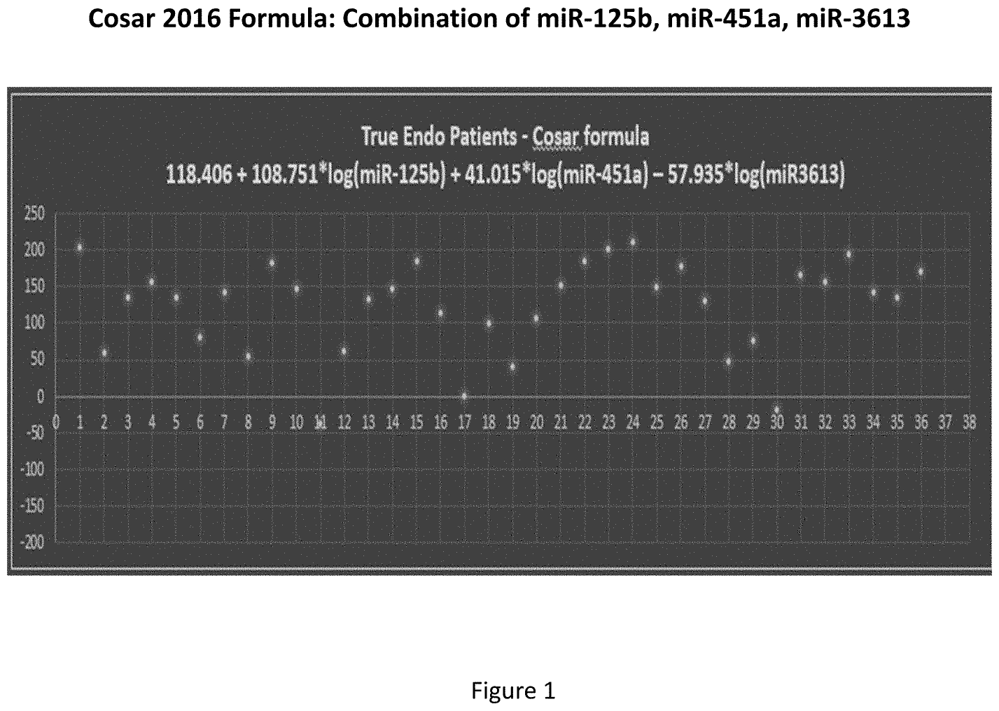

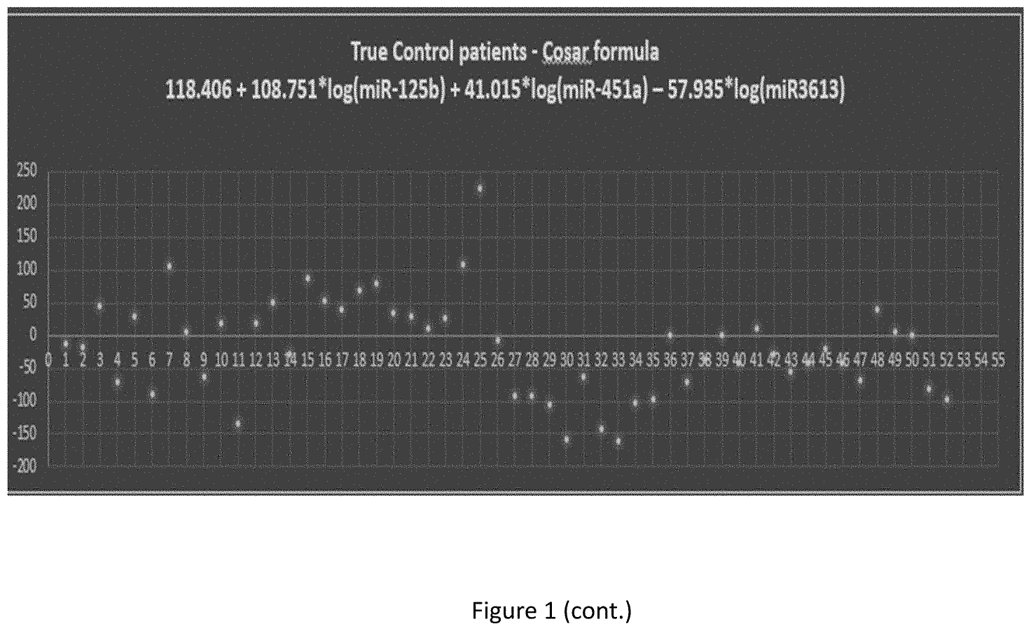

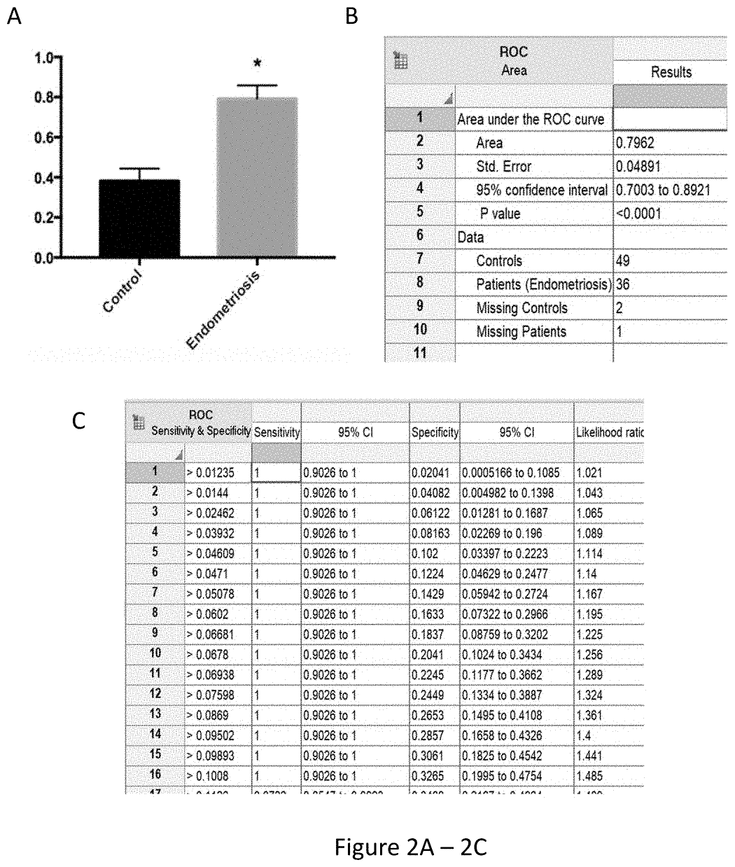

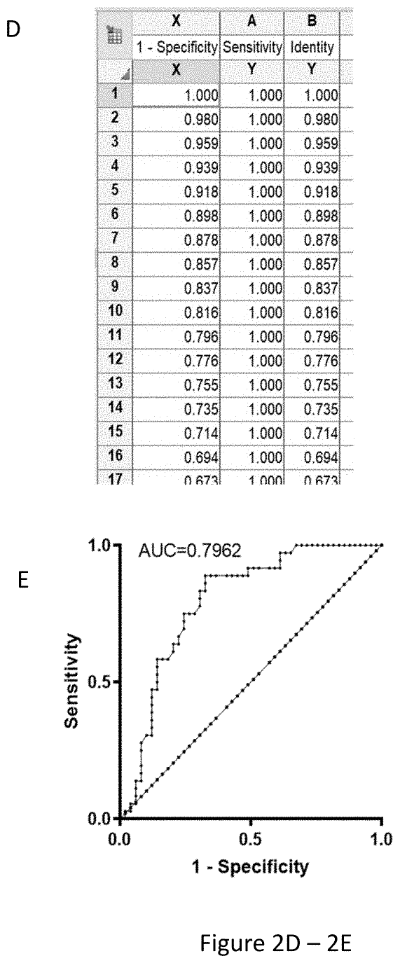

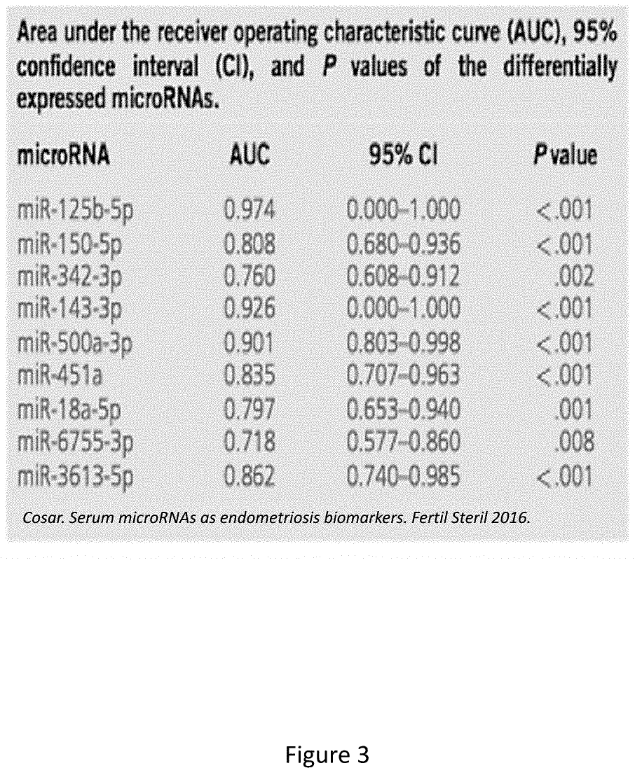

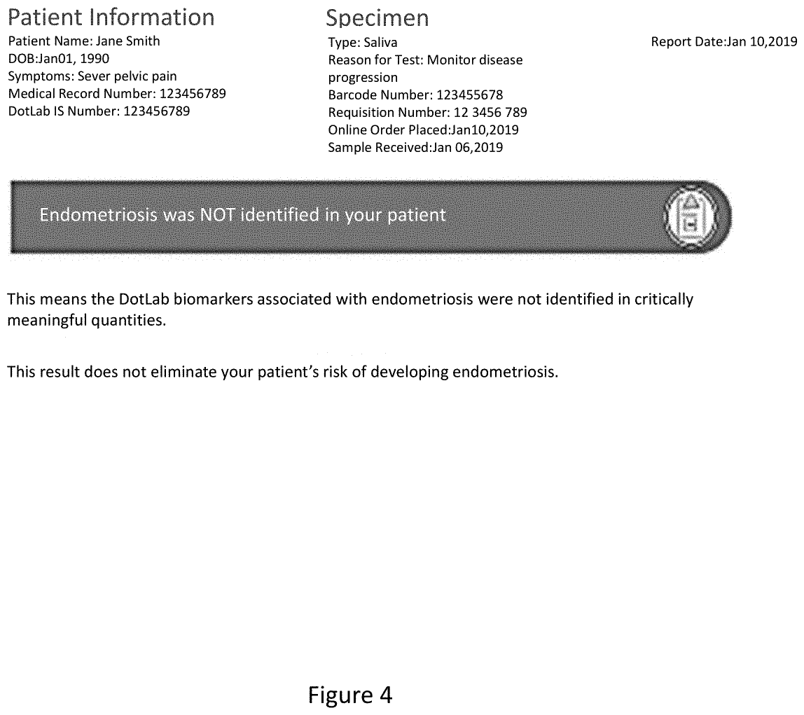

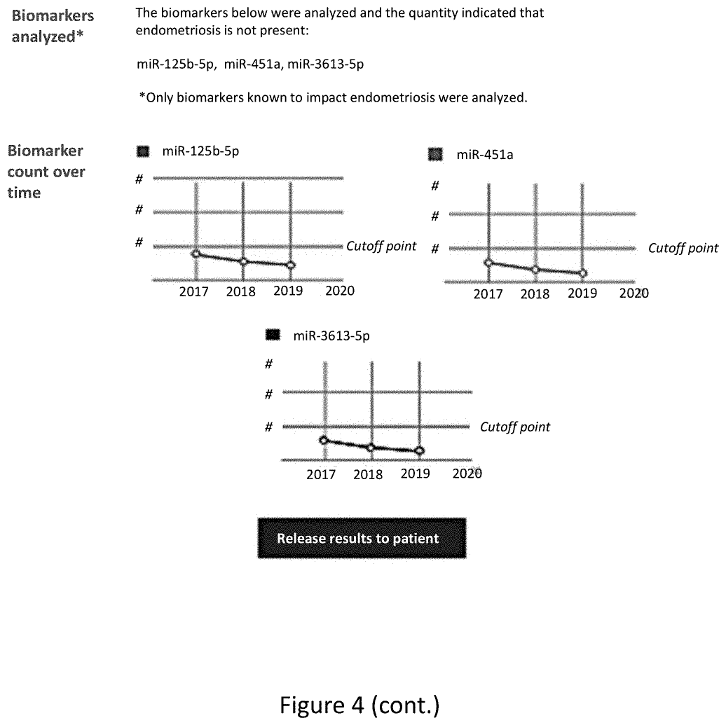

### DETAILED DESCRIPTION

There remains a need in the art for specific combinations of biomarkers that can be used to improve accuracy and yield a test with better sensitivity and specificity than any currently available biomarkers. miRNAs can be found in a wide array of bodily fluids such as tears, saliva, cerebrospinal fluid, pleural fluid, blood, urine, and peritoneal fluid. The wide diversity of bodily fluids from which miRNAs can be measured, many of which can be collected minimally-invasively, makes patient disease monitoring and diagnosis (e.g. in the case of endometriosis) using miRNAs a promising strategy for personalized medicine. The current disclosure addresses the need for improved combinations of biomarkers by providing improved combinations of miRNAs and associated methods thereof for detecting and monitoring endometriosis. Included in the present disclosure are methods, compositions, systems, kits, and assays for detecting endometriosis using one or more miRNA.

The present disclosure relates to the discovery that the expression level of particular microRNAs (miRNAs) is associated with endometriosis. Thus, in various embodiments described herein, the methods of the disclosure relate to methods of diagnosing a subject as having endometriosis, methods of assessing a subject's risk of having or developing endometriosis, methods of assessing the severity of a subject's endometriosis, methods of stratifying a subject having endometriosis for assignment in a clinical trial, methods of treating a subject diagnosed as having or at risk of endometriosis and methods of monitoring endometriosis treatment in a subject. Thus, the disclosure relates to quantitative algorithms and methods useful for the detection and quantification of miRNAs for the diagnosis, assessment, and characterization of endometriosis in a subject in need thereof, based upon the expression level of at least one miRNA that is associated with endometriosis. The markers of the disclosure can be used to screen, diagnose, monitor the onset, monitor the progression, and assess the treatment of endometriosis. The markers of the disclosure can be used to establish and evaluate treatment plans.

In some embodiments, the miRNAs that are associated with endometriosis is a marker or biomarker of endometriosis. In various embodiments, the biomarkers of the disclosure include one or more miR-18, miR-18a, miR-18a-5p, miR-34c, miR-125, miR-125b, miR-125b-5p, miR-126, miR-135, miR-141, miR-145, miR-143, miR-143-3p, miR-145-5p, miR-150, miR-150-5p, miR-200, miR-200a, miR-200b, miR-214, miR-342, miR-342-3p, miR-449, miR-449a, miR-451, miR-451a, miR-500, miR-500a, miR-500a-3p, miR-553, miR-3613, miR-3613-5p, miR-4668, miR-6755, miR-6755-3p, and let-7b. In some embodiments, the biomarkers of the disclosure include the combination of miR-125b, miR-451, and miR-3613. In some embodiments, the biomarkers of the disclosure include the combination of miR-150, miR-342, miR-451 and let-7b.

In some embodiments, the disclosure provides a quantitative algorithm for predicting an individual's risk of developing endometriosis. In some embodiments, the quantitative algorithm of the disclosure can predict risk at a time when a prophylactic therapy can be administered such that the emergence of the disease is prevented.

In some embodiments, the markers of the disclosure are noninvasive biomarkers for endometriosis that allow for early detection of the disease without surgical procedures. For example, altered expression of specific miRNAs in the biological sample of the subject with endometriosis may correlate with other clinical parameters, such as pelvic pain, infertility, and disease recurrence. Therefore, the markers of the disclosure can be used as markers for prognosis and recurrence. This is an advantage because repeated surgical procedures used in the art for diagnosing endometriosis and related complications can be avoided.

The present disclosure provides for the use of biomarkers of endometriosis in a quantitative algorithm for the diagnosis and prognosis of endometriosis. Generally, the methods of this disclosure find use in diagnosing or for providing a prognosis for endometriosis by detecting the expression levels of biomarkers, which are differentially expressed (up- or down-regulated) in blood, plasma or serum from a patient. Similarly, these markers can be used to diagnose reduced fertility in a patient with endometriosis or to provide a prognosis for a fertility trial in a patient suffering from endometriosis. The markers according to the present disclosure can be used to identify a subject in need of a treatment for endometriosis, and therefore, in some embodiments, the present disclosure provides methods of treating endometriosis based on the results of the diagnostic assay. The present disclosure also provides methods of identifying a compound for treating or preventing endometriosis. The present disclosure provides kits for the diagnosis or prognosis of endometriosis.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” refers to one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

“Antisense,” as used herein, refers to a nucleic acid sequence which is complementary to a target sequence, such as, by way of example, complementary to a target miRNA sequence, including, but not limited to, a mature target miRNA sequence, or a sub-sequence thereof. Typically, an antisense sequence is fully complementary to the target sequence across the full length of the antisense nucleic acid sequence.

The term “body fluid” or “bodily fluid” as used herein refers to any fluid from the body of an animal. Examples of body fluids include, but are not limited to, plasma, serum, blood, lymphatic fluid, cerebrospinal fluid, synovial fluid, urine, saliva, mucous, phlegm, sputum, tears, pleural fluid, and peritoneal fluid. A body fluid sample may be collected by any suitable method. The body fluid sample may be used immediately or may be stored for later use. Any suitable storage method may be used to store the body fluid sample: for example, the sample may be frozen at about −20° C. to about −70° C. Suitable body fluids are acellular fluids. “Acellular” fluids include body fluid samples in which cells are absent or are present in such low amounts that the miRNA level determined reflects its level in the liquid portion of the sample, rather than in the cellular portion. Such acellular body fluids are generally produced by processing a cell-containing body fluid by, for example, centrifugation or filtration, to remove the cells. Typically, an acellular body fluid contains no intact cells or very minimal amounts of cells; however, some acellular body fluids may contain cell fragments or cellular debris. Examples of acellular fluids include plasma or serum, or body fluids from which cells have been removed.

As used herein, the term “cell-free” refers to the condition of the nucleic acid as it appeared in the body directly before the sample is obtained from the body. For example, nucleic acids may be present in a body fluid such as blood or saliva in a cell-free state in that they are not associated with a cell. However, the cell-free nucleic acids may have originally been associated with a cell, such as an endometrial cell before entering the bloodstream or other body fluid. In contrast, nucleic acids that are solely associated with cells in the body are generally not considered to be “cell-free.” For example, nucleic acids extracted from a cellular sample are generally not considered “cell-free” as the term is used herein.

The term “clinical factors” as used herein, refers to any data that a medical practitioner may consider in determining a diagnosis or prognosis of disease. Such factors include, but are not limited to, the patient's medical history, a physical examination of the patient, complete blood count, analysis of the activity of enzymes, examination of cells, cytogenetics, and immunophenotyping of blood cells.

“Complementary” as used herein refers to the broad concept of subunit sequence complementarity between two nucleic acids. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, preferably at least about 60% and more preferably at least about 80% of corresponding positions in each of the molecules are occupied by nucleotides which normally base pair with each other (e.g., A:T and G:C nucleotide pairs).

As used herein, the term “diagnosis” refers to detecting a disease or disorder or determining the stage or degree of a disease or disorder. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease may not be exclusively related to the particular disease; i.e. there may be differential diagnoses that can be inferred from a diagnostic factor or symptom. Likewise, there may be instances where a factor or symptom that is indicative of a particular disease is present in an individual that does not have the particular disease. The diagnostic methods may be used independently, or in combination with other suitable diagnosing and/or staging methods.

As used herein, the phrase “difference of the level” refers to differences in the quantity of a particular marker, such as a nucleic acid or a protein, in a sample as compared to a control or reference level. For example, the quantity of a particular biomarker may be present at an elevated amount or at a decreased amount in samples of patients with a disease compared to a reference level. In some embodiments, a “difference of a level” may be a difference between the quantity of a particular biomarker present in a sample as compared to a control of at least about 1%, at least about 2%, at least about 3%, at least about 5%, at least about 10%, at least about 15%, at least about 20%, at least about 25%, at least about 30%, at least about 35%, at least about 40%, at least about 50%, at least about 60%, at least about 75%, at least about 80% or more. In some embodiments, a “difference of a level” may be a statistically significant difference between the quantity of a biomarker present in a sample as compared to a control. For example, a difference may be statistically significant if the measured level of the biomarker falls outside of about 1.0 standard deviations, about 1.5 standard deviations, about 2.0 standard deviations, or about 2.5 stand deviations of the mean of any control or reference group.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

The term “comparator” describes a material comprising none, or a normal, low, or high level of one of more of the marker (or biomarker) expression products of one or more the markers (or biomarkers) of the disclosure, such that the comparator may serve as a control or reference standard against which a sample can be compared.

The terms “dysregulated” and “dysregulation” as used herein describes a decreased (down-regulated) or increased (up-regulated) level of expression of a miRNA present and detected in a sample obtained from subject as compared to the level of expression of that miRNA in a comparator sample, such as a comparator sample obtained from one or more normal, not-at-risk subjects, or from the same subject at a different time point. In some instances, the level of miRNA expression is compared with an average value obtained from more than one not-at-risk individuals. In other instances, the level of miRNA expression is compared with a miRNA level assessed in a sample obtained from one normal, not-at-risk subject.

By the phrase “determining the level of marker (or biomarker) expression” is meant an assessment of the degree of expression of a marker in a sample at the nucleic acid or protein level, using technology available to the skilled artisan to detect a sufficient portion of any marker expression product.

The terms “determining,” “measuring,” “assessing,” and “assaying” are used interchangeably and include both quantitative and qualitative measurement, and include determining if a characteristic, trait, or feature is present or not. Assessing may be relative or absolute. “Assessing the presence of” includes determining the amount of something present, as well as determining whether it is present or absent.

“Differentially increased expression” or “up regulation” refers to expression levels which are at least 10% or more, for example, 20%, 30%, 40%, or 50%, 60%, 70%, 80%, 90% higher or more, and/or 1.1-fold, 1.2-fold, 1.4 fold, 1.6 fold, 1.8 fold, 2.0 fold higher or more, and any and all whole or partial increments there between than a comparator.

“Differentially decreased expression” or “down regulation” refers to expression levels which are at least 10% or more, for example, 20%, 30%, 40%, or 50%, 60%, 70%, 80%, 90% lower or less, and/or 2.0-fold, 1.8-fold, 1.6 fold, 1.4 fold, 1.2 fold, 1.1 fold or less lower, and any and all whole or partial increments there between than a comparator.

As used herein “endogenous” refers to any material from or produced inside an organism, cell, tissue or system.

The term “expression” as used herein is defined as the transcription and/or translation of a particular nucleotide sequence.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity.” “Inhibitors,” “activators,” and “modulators” of the markers are used to refer to activating, inhibitory, or modulating molecules identified using in vitro and in vivo assays of endometriosis biomarkers. Inhibitors are compounds that, e.g., bind to, partially or totally block activity, decrease, prevent, delay activation, inactivate, desensitize, or down regulate the activity or expression of endometriosis biomarkers.

“Activators” are compounds that increase, open, activate, facilitate, enhance activation, sensitize, agonize, or up regulate activity of endometriosis biomarkers, e.g., agonists.

Inhibitors, activators, or modulators also include genetically modified versions of endometriosis biomarkers, e.g., versions with altered activity, as well as naturally occurring and synthetic ligands, antagonists, agonists, antibodies, peptides, cyclic peptides, nucleic acids, antisense molecules, ribozymes, RNAi, microRNA, and siRNA molecules, small organic molecules and the like. Such assays for inhibitors and activators include, e.g., expressing endometriosis biomarkers in vitro, in cells, or cell extracts, applying putative modulator compounds, and then determining the functional effects on activity, as described elsewhere herein.

As used herein, an “instructional material” includes a publication, a recording, a diagram, or any other medium of expression which can be used to communicate the usefulness of a compound, composition, vector, method or delivery system of the disclosure in the kit for effecting alleviation of the various diseases or disorders recited herein. Optionally, or alternately, the instructional material can describe one or more methods of alleviating the diseases or disorders in a cell or a tissue of a mammal. The instructional material of the kit of the disclosure can, for example, be affixed to a container which contains the identified compound, composition, vector, or delivery system of the disclosure or be shipped together with a container which contains the identified compound, composition, vector, or delivery system. Alternatively, the instructional material can be shipped separately from the container with the intention that the instructional material and the compound be used cooperatively by the recipient.

As used herein, “isolated” denotes altered or removed from the natural state through the actions, directly or indirectly, of a human being. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. “Measuring” or “measurement,” or alternatively “detecting” or “detection,” denotes assessing the presence, absence, quantity or amount (which can be an effective amount) of either a given substance within a clinical or subject-derived sample, including the derivation of qualitative or quantitative concentration levels of such substances, or otherwise evaluating the values or categorization of a subject's clinical parameters.

As used herein, “microRNA” or “miRNA” describes small non-coding RNA molecules, generally about 15 to about 50 nucleotides in length, preferably 17-23 nucleotides, which can play a role in regulating gene expression through, for example, a process termed RNA interference (RNAi). RNAi describes a phenomenon whereby the presence of an RNA sequence that is complementary or antisense to a sequence in a target gene messenger RNA (mRNA) results in inhibition of expression of the target gene. miRNAs are processed from hairpin precursors of about 70 or more nucleotides (pre-miRNA) which are derived from primary transcripts (pri-miRNA) through sequential cleavage by RNAse III enzymes. miRBase is a comprehensive microRNA database located at mirbase.org, incorporated by reference herein in its entirety for all purposes.

A “mutation,” as used herein, refers to a change in nucleic acid or polypeptide sequence relative to a reference sequence (which is preferably a naturally-occurring normal or “wild-type” sequence), and includes translocations, deletions, insertions, and substitutions/point mutations. A “mutant,” as used herein, refers to either a nucleic acid or protein comprising a mutation.

“Naturally occurring” as used herein describes a composition that can be found in nature as distinct from being artificially produced. For example, a nucleotide sequence present in an organism, which can be isolated from a source in nature and which has not been intentionally modified by a person, is naturally occurring.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand.” Sequences on the DNA strand which are located 5′ to a reference point on the DNA are referred to as “upstream sequences.” Sequences on the DNA strand which are 3′ to a reference point on the DNA are referred to as “downstream sequences.”

As used herein, “polynucleotide” includes cDNA, RNA, DNA/RNA hybrid, anti-sense RNA, siRNA, miRNA, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the disclosure are alterations of a wild type or synthetic gene, including but not limited to deletion, insertion, substitution of one or more nucleotides, or fusion to other polynucleotide sequences.

As used herein, a “primer” for amplification is an oligonucleotide that specifically anneals to a target or marker nucleotide sequence. The 3′ nucleotide of the primer is optimally substantially identical to the target or marker sequence at a corresponding nucleotide position for optimal primer extension by a polymerase. As used herein, a “forward primer” is a primer that anneals to the anti-sense strand of double stranded DNA (dsDNA). A “reverse primer” anneals to the sense-strand of dsDNA.

The term “recombinant DNA” as used herein is defined as DNA produced by joining pieces of DNA from different sources.

As used herein, the term “providing a prognosis” refers to providing a prediction of the probable course and outcome of endometriosis, including prediction of severity, duration, chances of recovery, etc. The methods can also be used to devise a suitable therapeutic plan, e.g., by indicating whether or not the condition is still at an early stage or if the condition has advanced to a stage where aggressive therapy would be ineffective.

A “reference level” of a biomarker denotes a level of the biomarker that is indicative of a particular disease state, phenotype, or lack thereof, as well as combinations of disease states, phenotypes, or lack thereof. A “positive” reference level of a biomarker denotes a level that is indicative of a particular disease state or phenotype. A “negative” reference level of a biomarker refers to a level that is indicative of a lack of a particular disease state or phenotype.

“Sample” or “biological sample” as used herein refers to a biological material isolated from an individual. The biological sample may contain any biological material suitable for detecting the biomarkers according to the disclosure and may comprise cellular and/or non-cellular material obtained from the individual.

“Standard control value” as used herein refers to a predetermined amount of a particular protein or nucleic acid that is detectable in a biological sample. The standard control value is suitable for the use of a method of the present disclosure, in order for comparing the amount of a protein or nucleic acid of interest that is present in a biological sample. An established sample serving as a standard control provides an average amount of the protein or nucleic acid of interest in the biological sample that is typical for an average, healthy person of reasonably matched background, e.g., gender, age, ethnicity, and medical history. A standard control value may vary depending on the protein or nucleic acid of interest and the nature of the sample (e.g., serum).

The terms “subject,” “patient,” “individual,” and the like are used interchangeably herein, and refer to any animal, or cells thereof whether in vitro or in situ, amenable to the methods described herein. In certain non-limiting embodiments, the patient, subject or individual is a human.

The terms “underexpress,” “underexpression,” “underexpressed,” or “down-regulated” interchangeably refer to a protein or nucleic acid that is transcribed or translated at a detectably lower level in a biological sample from a woman with endometriosis, in comparison to a biological sample from a woman without endometriosis. The term includes underexpression due to transcription, post transcriptional processing, translation, post-translational processing, cellular localization (e.g., organelle, cytoplasm, nucleus, cell surface), and RNA and protein stability, as compared to a control. Underexpression can be detected using techniques for detecting mRNA (e.g., Q-PCR, RT-PCR, PCR, hybridization) or proteins (e.g., ELISA, immunohistochemical techniques). Underexpression can be 10%, 20%, 30%, 40%, 50%, 60%, 70%, 80%, 90% or less in comparison to a control. In certain instances, underexpression is 1-, 2-, 3-, 4-, 5-, 6-, 7-, 8-, 9-, 10-fold or more lower levels of transcription or translation in comparison to a control.

The terms “overexpress,” “overexpression,” “overexpressed,” or “up-regulated” interchangeably refer to a protein or nucleic acid (RNA) that is transcribed or translated at a detectably greater level, usually in a biological sample from a woman with endometriosis, in comparison to a biological sample from a woman without endometriosis. The term includes overexpression due to transcription, post transcriptional processing, translation, post-translational processing, cellular localization (e.g., organelle, cytoplasm, nucleus, cell surface), and RNA and protein stability, as compared to a cell from a woman without endometriosis. Overexpression can be detected using techniques for detecting mRNA (e.g., Q-PCR, RT-PCR, PCR, hybridization) or proteins (e.g., ELISA, immunohistochemical techniques). Overexpression can be 10%, 20%, 30%, 40%, 50%, 60%, 70%, 80%, 90% or more in comparison to a cell from a woman without endometriosis. In certain instances, overexpression is 1-, 2-, 3-, 4-, 5-, 6-, 7-, 8-, 9-, 10-fold, or more higher levels of transcription or translation in comparison to a cell from a woman without endometriosis.

“Variant” as the term is used herein, is a nucleic acid sequence or a peptide sequence that differs in sequence from a reference nucleic acid sequence or peptide sequence respectively but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be naturally occurring or non-naturally occurring. Non-naturally occurring variants of nucleic acids and peptides may be made by mutagenesis techniques or by direct synthesis.

As used herein, the terms “treat,” “ameliorate,” “treatment,” and “treating” are used interchangeably. These terms refer to an approach for obtaining beneficial results including, but are not limited to, therapeutic benefit and/or a prophylactic benefit. Therapeutic benefit refers to eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, treatment may be administered to a patient at risk of developing a particular disease, or to a patient reporting one or more of the physiological symptoms of a disease, even though a diagnosis of this disease may not have been made.

The term “or” as used herein and throughout the disclosure, generally denotes “and/or” unless the context dictates otherwise.

As used herein, the term “circulating miRNA” refers to any miRNA in a body fluid, regardless of whether the body fluid is traditionally considered to be a part of the circulatory system. For example, “circulating miRNA” would encompass miRNA present in a subject's blood and would also encompass miRNA present in a subject's saliva, urine, or other bodily fluid.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present disclosure relates to the discovery that the level of expression or expression pattern of particular biomarkers is associated with endometriosis. In some embodiments, one or more biomarkers associated with endometriosis are up-regulated, or expressed at a higher than normal level. In some embodiments, one or more biomarkers associated with endometriosis are down-regulated, or expressed at a lower than normal level. Thus, the disclosure relates to compositions and methods useful for the diagnosis, detection, assessment, and characterization of endometriosis in a subject in need thereof, based upon the expression level or expression pattern of one or more biomarkers that is associated with endometriosis.

In exemplary embodiments, the miRNAs are selected from the group consisting of: miR-18, miR-18a, miR-18a-5p, miR-34c, miR-125, miR-125b, miR-125b-5p, miR-126, miR-135, miR-141, miR-143, miR-143-3p, miR-145, miR-145-5p, miR-150, miR-150-5p, miR-200, miR-200a, miR-200b, miR-214, miR-342, miR-342-3p, miR-449, miR-449a, miR-451, miR-451a, miR-500, miR-500a, miR-500a-3p, miR-553, miR-3613, miR-3613-5p, miR-4668, miR-6755, miR-6755-3p, and let-7b, and any combination thereof. In some embodiments, the present disclosure relates to a screening assay of a subject to determine whether the subject has an elevated level of expression one or more of miR-125, miR-150, miR-342, and miR-451. In some embodiments, the present disclosure relates to a screening assay of a subject to determine whether the subject has a reduced level of expression of one or more of miR-3613 and let-7b. In some embodiments, a combination of biomarker levels of the disclosure useful for diagnosing endometriosis include the combination of an increased level of miR-125, an increased level of miR-451, and a decreased level of miR-3613. In some embodiments, a combination of biomarker levels of the disclosure useful for diagnosing endometriosis include the combination of an increased level of miR-150, an increased level of miR-342, an increased level of miR-451 and a decreased level of let-7b. Sequences of the miRNA family members are publicly available from miRBase at (mirbase.org).

In one aspect, the methods generally provide for the detection, measuring, and comparison of a pattern of circulating miRNA in a patient sample. In other aspects, the methods generally provide for detection, measuring and comparison of a pattern of miRNA present in a sample of bodily fluid (e.g., blood, plasma, serum, saliva, urine). In the context of endometriosis, it is frequently difficult to have access to the diseased cells.

As such, a method that detects endometriosis using a relatively non-invasive method such as a blood draw or collection of saliva would be very beneficial. In various embodiments, the present methods overcome problems of cancer diagnosis by determining the levels of miRNAs in the plasma of patients with liver diseases. An alteration (i.e., an increase or decrease) in the level of a miRNA gene product in the sample obtained from the subject, relative to the level of a corresponding miRNA gene product in a control sample, is indicative of the presence of endometriosis in the subject. In some embodiments, the level of at least one miRNA gene product in the test sample is greater than the level of the corresponding miRNA gene product in the control sample. In some embodiments, the level of at least one miRNA gene product in the test sample is less than the level of the corresponding miRNA gene product in the control sample.

In some embodiments, the disclosure provides a method for detecting at least one, at least two, at least three, at least four, at least five, or at least ten of miR-125, miR-150, miR-342, miR-145, miR-143, miR-500, miR-451, miR-18, miR-214, miR-126, miR-6755, miR-3613, miR-553, miR-4668, let-7b, miR-135, miR-449a, miR-34c, miR-200a, miR-200b, miR-141, miR-125b-5p, miR-150-5p, miR-342-3p, miR-145-5p, miR-143-3p, miR-500a-3p, miR-451a, miR-18a-5p, miR-6755-3p, miR-3613-5p.

Additional diagnostic markers may be combined with the circulating miRNA level to construct models for predicting the presence or absence or stage of a disease. For example, clinical factors of relevance to the diagnosis of endometriosis diseases, include, but are not limited to, the patient's medical history, a physical examination, and other biomarkers. A diagnosis of endometriosis may also be informed by a patient's symptoms, including the type of symptoms, duration of symptoms and degree of symptoms.

Accordingly, the disclosure may provide a new and convenient platform for diagnosing endometriosis, often with relatively high sensitivity. In some embodiments, the system or methods of the disclosure provides a platform for diagnosing endometriosis with at least 80% sensitivity, preferably at least 90% sensitivity, preferably at least 91% sensitivity, preferably at least 92% sensitivity, preferably at least 93% sensitivity, preferably at least 94% sensitivity, preferably at least 95% sensitivity, preferably at least 96% sensitivity, preferably at least 97% sensitivity, preferably at least 98% sensitivity, preferably at least 99% sensitivity, or preferably 100% sensitivity.

In some embodiments, the system or methods of the disclosure provides a platform for diagnosing endometriosis. In some embodiments, the system of the disclosure provides a platform for diagnosing endometriosis with at least 80% specificity, preferably at least 90% specificity, preferably at least 91% specificity, preferably at least 92% specificity, preferably at least 93% specificity, preferably at least 94% specificity, preferably at least 95% specificity, preferably at least 96% specificity, preferably at least 97% specificity, preferably at least 98% specificity, preferably at least 99% specificity, or preferably 100% specificity.

In some embodiments, the disclosure provides a system or methods for diagnosing endometriosis, with at least 90%, 91%, 92%, 93%, 94%, 95%, 96%, 97%, 98%, 99%, 100% sensitivity; at least 90%, 91%, 92%, 93%, 94%, 95%, 96%, 97%, 98%, 99%, 100% specificity; or both at least 90%, 91%, 92%, 93%, 94%, 95%, 96%, 97%, 98%, 99%, 100% sensitivity and specificity. In some embodiments, the disclosure provides a system or method for diagnosing endometriosis with at least 90%, 91%, 92%, 93%, 94%, 95%, 96%, 97%, 98%, 99%, 100% accuracy.

In some embodiments, this disclosure provides a combination of miRNA (e.g., a combination of three miRNAs such as miR-125b, miR-451a, and miR-3613, a combination of four miRNA, etc.), that yield an AUC score of at least 0.85, at least 0.90, at least 0.91, at least 0.92, at last 0.93, at least 0.94, at least 0.95, or at least 0.97. In some cases, the AUC is obtained after using a logistic regression analysis.

Generally, the methods of this disclosure find use in diagnosing or for providing a prognosis for endometriosis by detecting the expression levels of biomarkers, which are differentially expressed (up- or down-regulated) in blood or serum from a patient. These markers can be used to distinguish the stage or severity of endometriosis. These markers can also be used to provide a prognosis for the course of treatment in a patient with endometriosis. Similarly, these markers can be used to diagnose infertility in a patient with endometriosis or to provide a prognosis for a fertility trial in a patient suffering from endometriosis. The biomarkers of the present disclosure can be used alone or in combination for the diagnosis or prognosis of endometriosis.

In some embodiments, the methods of the present disclosure find use in assigning treatment to a patient suffering from endometriosis. By detecting the expression levels of biomarkers found herein, the appropriate treatment can be assigned to a patient suffering from endometriosis. These treatments can include, but are not limited to, hormone therapy (e.g., administration of oral contraceptives) and surgical treatment. Similarly, the methods of the current disclosure can be used to assign treatment to a patient with reduced fertility due to endometriosis. In this fashion, by determining the degree to which the patient's fertility has been reduced, through the detection of biomarkers found herein, the appropriate treatment can be assigned. Relevant treatments include, but are not limited to, hormone therapy, chemotherapy, immunotherapy, and surgical treatment.

Diagnostic and prognostic kits comprising one or more markers for use are provided herein. Also provided by the disclosure are methods for identifying compounds that are able to prevent or treat endometriosis or reduced fertility caused by endometriosis by modulating the expression level or activity of markers found in any one of the identified gene subsets. Therapeutic methods are provided, wherein endometriosis or reduced fertility caused by endometriosis is treated using an agent that targets the markers of the disclosure.

In various embodiments, the methods of the disclosure relate to methods of assessing a subject's risk of having or developing endometriosis, methods of assessing the severity of a subject's endometriosis, methods of diagnosing endometriosis, methods of characterizing endometriosis, and methods of stratifying a subject having endometriosis in a clinical trial.

In various embodiments, the methods according to the disclosure relate to methods of diagnosing, treating or preventing endometriosis or a disease or disorder associated with endometriosis in a subject, where the subject has been identified as having a differentially expressed level of one or more biomarkers associated with endometriosis. In some embodiments, the treatment comprises administering a pharmaceutical composition for the treatment or prevention of endometriosis. Exemplary pharmaceutical compositions that can be administered include but are not limited to oral contraceptives. In some embodiments, the treatment comprises administering a disease-specific therapy for the treatment or prevention of endometriosis. In some embodiments, the treatment comprises performing a surgical intervention for the treatment or prevention of endometriosis. In some embodiments, the methods according to the disclosure relate to altering a treatment in a subject who has been identified as not having a differentially expressed level of one or more biomarkers associated with endometriosis by discontinuing a disease-specific therapy.

In some embodiments, the present disclosure relates to methods of diagnosing, treating or preventing endometriosis. In some embodiments, the methods according to the present disclosure relate to methods of diagnosing, treating or preventing a disease or disorder associated with endometriosis. Diseases and disorders associated with endometriosis include, but are not limited to, cancer, autoimmune diseases, asthma or allergic manifestations, and cardiovascular disease.

### Sample Preparation

Test samples of acellular body fluid or cell-containing samples may be obtained from an individual or patient. Methods of obtaining test samples include but are not limited to aspirations or drawing of blood or other fluids. Samples may include, but are not limited to, whole blood, serum, plasma, saliva, cerebrospinal fluid (CSF), pericardial fluid, pleural fluid, urine, and eye fluid. In some embodiments in which the test sample contains cells, the cells may be removed from the liquid portion of the sample by suitable methods (e.g., centrifugation) to yield acellular body fluid. In suitable embodiments, serum or plasma are used as the acellular body fluid sample. Plasma and serum can be prepared from whole blood using suitable methods. In these embodiments, data may be normalized by volume of acellular body fluid.

In some embodiments, test samples of saliva may be obtained from a subject. Methods of obtaining saliva samples may include but are not limited to forcible ejection from the subject's mouth (e.g., spitting), aspiration, or removal by a swab or other collection tool. As used herein, the term “saliva” does not include sputum, since sputum pertains to mucus or phlegm samples. In some embodiments, the saliva may be separated into cellular and non-cellular fractions by suitable methods (e.g., centrifugation). In some embodiments, nucleic acids may be extracted from the cellular or non-cellular fractions.

Variability in sample preparation of cell-containing samples can be corrected by normalizing the data by, for example, protein content or cell number. In certain embodiments, the sample may be normalized relative to the total protein content in the sample. Total protein content in the sample can be determined using standard procedures, including, without limitation, Bradford assay and the Lowry method. In some embodiments, the sample may be normalized relative to cell number.

In various embodiments, the subject is a human subject, and may be of any race. In some embodiments, the subject is a postpubescent female subject. Representative subjects include those who are suspected of having endometriosis, those who have been diagnosed with endometriosis, those who have a family history of endometriosis, or those who are at higher risk of developing endometriosis, including, but not limited to female subjects having immune system disorders, abdominal surgery, uterine growths, structural abnormality of the uterus, cervix or vagina, or subjects who have early, frequent or prolonged menstrual periods.

In various embodiments, the test sample is a biological sample (e.g., fluid, tissue, cell, cellular component, etc.) of the subject containing at least a fragment of a gene product (e.g., polypeptide or mRNA) of one or more of miR-125, miR-150, miR-342, miR-145, miR-143, miR-500, miR-451, miR-18, miR-214, miR-126, miR-6755, miR-3613, miR-553, miR-4668, let-7b, miR-135, miR-449a, miR-34c, miR-200a, miR-200b, miR-141, miR-125b-5p, miR-150-5p, miR-342-3p, miR-145-5p, miR-143-3p, miR-500a-3p, miR-451a, miR-18a-5p, miR-6755-3p, or miR-3613-5p. The biological sample can be a sample from any source which contains nucleic acid, such as a fluid, tissue, cell, cellular component, or a combination thereof. A biological sample can be obtained by appropriate methods, such as, by way of examples, blood draw, fluid draw, or biopsy. A biological sample can be used as the test sample; alternatively, a biological sample can be processed to enhance access to the nucleic acids, or copies of the nucleic acids, and the processed biological sample can then be used as the test sample. For example, in various embodiments, an amplification method can be used to amplify nucleic acids comprising all or a fragment of a miRNA in a biological sample, for use as the test sample in the assessment of the level in the biological sample. In some embodiments, the biological sample is blood, plasma, saliva, or urine. In some embodiments, the biological sample is blood.

Methods

The present disclosure provides methods for assessing the risk for endometriosis in a subject. The present disclosure includes methods for identifying subjects who have an increased or enhanced risk for endometriosis and subjects who do not have an enhanced risk for endometriosis by detection of the biomarkers disclosed herein. These biomarkers also are useful for monitoring subjects undergoing treatments and therapies for endometriosis. These biomarkers also are useful for selecting or modifying therapies and treatments that would be efficacious in subjects having endometriosis and subjects who have had endometriosis.

The disclosure provides improved methods for assessing the risk for endometriosis. The risk for endometriosis can be assessed by measuring one or more of the biomarkers described herein, and comparing the measured values to comparator values, reference values, or index values. Such a comparison can be undertaken with mathematical algorithms or formula in order to combine information from results of multiple individual biomarkers and other parameters into a single measurement or index. For example, in certain embodiments, the comparison is undertaken with a quantitative algorithm, as described elsewhere herein.

Subjects identified as having an enhanced risk for endometriosis can optionally be selected to receive treatment regimens, such as laparoscopic surgery or administration of therapeutic compounds to prevent, treat or delay the occurrence, reoccurrence or progression of endometriosis.

Identifying a subject as having an enhanced risk for endometriosis recurrence after laparoscopy allows for the selection and initiation of various therapeutic interventions or treatment regimens in order to delay, reduce or prevent recurrence in those at risk. Further, identifying a subject with a low risk, or those who do not have an enhanced risk, for endometriosis recurrence allows for the sparing of unnecessary additional therapy administered to the subject.

Monitoring the levels of at least one biomarker also allows for a course of treatment to be monitored. For example, a sample can be provided from a subject undergoing treatment regimens or therapeutic interventions. Such treatment regimens or therapeutic interventions can include surgery, administration of pharmaceutical compositions, and the like.

The biomarkers of the present disclosure can thus be used to generate a biomarker profile or signature of the subjects: (i) who have an increased risk for endometriosis, (ii) who do not have an increased risk for endometriosis, and/or (iii) who have a low risk for endometriosis. The biomarker profile of a subject can be compared to a predetermined or comparator biomarker profile or reference biomarker profile to assess the risk for endometriosis. Data concerning the biomarkers of the present disclosure can also be combined or correlated with other data or test results, such as, without limitation, measurements of clinical parameters or other algorithms for endometriosis. Other data includes age, ethnicity, and other genomic data or protein expression data, specifically expression values of other gene signatures relevant to endometriosis outcomes, and the like. The data may also comprise subject information such as medical history and any relevant family history.

The present disclosure also provides methods for identifying agents for treating endometriosis that are appropriate or otherwise customized for a specific subject.

In this regard, a test sample from a subject, exposed to a therapeutic agent or a drug, can be taken and the level of one or more biomarkers can be determined. The level of one or more biomarkers can be compared to a sample derived from the subject before and after treatment or can be compared to samples derived from one or more subjects who have shown improvements in risk factors as a result of such treatment or exposure.

### Assays and Methods of Diagnosis

The present disclosure relates to the discovery that the expression level of particular miRNAs is associated with the presence, development, progression and severity of endometriosis. In various embodiments, the disclosure relates to a genetic screening assay of a subject to determine the level of expression of at least one miRNA associated with endometriosis in the subject. The present disclosure provides methods of assessing level of at least one miRNA associated with endometriosis, as well as methods of diagnosing a subject as having, or as being at risk of developing, endometriosis based upon the level of expression of at least one miRNA associated with endometriosis. In some embodiments, the diagnostic assays described herein are in vitro assays.

In some embodiments, the method of the disclosure is a diagnostic assay for assessing the presence, development, progression and/or severity of endometriosis in a subject in need thereof, by determining whether the level of at least one miRNA associated with endometriosis is decreased in a biological sample obtained from the subject. In various embodiments, to determine whether the level of the at least one miRNA associated with endometriosis is decreased in a biological sample obtained from the subject, the level of the at least one miRNA is compared with the level of at least one comparator control, such as a positive control, a negative control, a normal control, a wild-type control, a historical control, a historical norm, or the level of another reference molecule in the biological sample. In some embodiments, the diagnostic assay of the disclosure is an in vitro assay. In some embodiments, the diagnostic assay of the disclosure is an in vivo assay. In some embodiments, the method further comprising the step of treating the subject for endometriosis based on the results of the diagnostic assay.

In some embodiments, the present disclosure relates to a screening assay of a subject to determine whether the subject has a differentially expressed level or pattern of one or more of miR-125, miR-150, miR-342, miR-145, miR-143, miR-500, miR-451, miR-18, miR-214, miR-126, miR-6755, miR-3613, miR-553, miR-4668, let-7b, miR-135, miR-449a, miR-34c, miR-200a, miR-200b, miR-141, miR-125b-5p, miR-150-5p, miR-342-3p, miR-145-5p, miR-143-3p, miR-500a-3p, miR-451a, miR-18a-5p, miR-6755-3p, or miR-3613-5p. In some embodiments, the disclosure relates to a screening assay of a subject to determine whether the subject has an elevated level of expression one or more of miR-125, miR-150, miR-342, and miR-451. In some embodiments, the disclosure relates to a screening assay of a subject to determine whether the subject has a reduced level of expression of one or more of miR-3613 and let-7b. The present disclosure provides methods of assessing the level of at least one miRNA or pre-miRNA in a subject or a biological sample from a subject.

In some embodiments, the disclosure relates to diagnostic assays for diagnosing endometriosis in a subject, by determining whether the subject has a differentially expressed level of one or more of miR-125, miR-150, miR-342, miR-145, miR-143, miR-500, miR-451, miR-18, miR-214, miR-126, miR-6755, miR-3613, miR-553, miR-4668, let-7b, miR-135, miR-449a, miR-34c, miR-200a, miR-200b, miR-141, miR-125b-5p, miR-150-5p, miR-342-3p, miR-145-5p, miR-143-3p, miR-500a-3p, miR-451a, miR-18a-5p, miR-6755-3p, or miR-3613-5p. In some embodiments, the disclosure provides for a diagnostic assay for diagnosing endometriosis in a subject, by determining whether the subject has an elevated level of expression one or more of miR-125, miR-150, miR-342, and miR-451. In some embodiments, the disclosure provides for a diagnostic assay for diagnosing endometriosis in a subject, by determining whether the subject has a reduced level of expression of one or more of miR-3613 and let-7b. In some embodiments, the disclosure provides for a diagnostic assay for diagnosing endometriosis in a subject, by determining whether the subject has both a reduced level of expression of one or more of miR-3613 and let-7b and an elevated level of expression one or more of miR-125, miR-150, miR-342, and miR-451. In some embodiments, the method further comprising the step of treating the subject for endometriosis.

In various embodiments, to determine whether the level of expression of one or more of miR-125, miR-150, miR-342, miR-145, miR-143, miR-500, miR-451, miR-18, miR-214, miR-126, miR-6755, miR-3613, miR-553, miR-4668, let-7b, miR-135, miR-449a, miR-34c, miR-200a, miR-200b, miR-141, miR-125b-5p, miR-150-5p, miR-342-3p, miR-145-5p, miR-143-3p, miR-500a-3p, miR-451a, miR-18a-5p, miR-6755-3p, or miR-3613-5p is increased or reduced in a biological sample of the subject, the level of expression of the one or more of miR-125, miR-150, miR-342, miR-145, miR-143, miR-500, miR-451, miR-18, miR-214, miR-126, miR-6755, miR-3613, miR-553, miR-4668, let-7b, miR-135, miR-449a, miR-34c, miR-200a, miR-200b, miR-141, miR-125b-5p, miR-150-5p, miR-342-3p, miR-145-5p, miR-143-3p, miR-500a-3p, miR-451a, miR-18a-5p, miR-6755-3p, or miR-3613-5p is compared with the level of at least one comparator control, such as a positive control, a negative control, a historical control, a historical norm, or the level of another reference molecule in the biological sample. The results of the diagnostic assay can be used alone, or in combination with other information from the subject, or other information from the biological sample obtained from the subject. In some embodiments, the method further comprising the step of treating the subject for endometriosis.

In various embodiments of the assays of the disclosure, the level of expression of the biomarker is determined to be elevated or increased when the level of expression of the biomarker is increased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator control.

In various embodiments of the methods according to the disclosure, the level of expression of the biomarker is determined to be elevated or increased when the level of expression of the biomarker in the biological sample is increased by at least 1-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 3.5-fold, at least 4-fold, at least 4.5-fold, at least 5-fold, at least 5.5-fold, at least 6-fold, at least 6.5-fold, at least 7-fold, at least 7.5-fold, at least 8-fold, at least 8.5-fold, at least 9-fold, at least 9.5-fold, at least 10-fold, at least 11-fold, at least 12-fold, at least 13-fold, at least 14-fold, at least 15-fold, at least 20-fold, at least 25-fold, at least 30-fold, at least 40-fold, at least 50-fold, at least 75-fold, at least 100-fold, at least 200-fold, at least 250-fold, at least 500-fold, or at least 1000-fold, when compared with a comparator.

In other various embodiments of the assays according to the disclosure, the level of expression of the biomarker is determined to be reduced or decreased when the level of expression of the biomarker is reduced or decreased by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator control.

In other various embodiments of the assays according to the disclosure, the level of expression of the biomarker is determined to be reduced or decreased when the level of expression of the biomarker is reduced or decreased by at least 1-fold, at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3-fold, at least 3.5-fold, at least 4-fold, at least 4.5-fold, at least 5-fold, at least 5.5-fold, at least 6-fold, at least 6.5-fold, at least 7-fold, at least 7.5-fold, at least 8-fold, at least 8.5-fold, at least 9-fold, at least 9.5-fold, at least 10-fold, at least 11-fold, at least 12-fold, at least 13-fold, at least 14-fold, at least 15-fold, at least 20-fold, at least 25-fold, at least 30-fold, at least 40-fold, at least 50-fold, at least 75-fold, at least 100-fold, at least 200-fold, at least 250-fold, at least 500-fold, or at least 1000-fold, when compared with a comparator.

In the assay methods of the disclosure, a test biological sample from a subject is assessed for the expression level of at least one miRNA associated with endometriosis. The test biological sample can be an in vitro sample or an in vivo sample. In various embodiments, the subject is a human subject, and may be of any race, sex and age. Representative subjects include those who are suspected of having endometriosis, those who have been diagnosed with endometriosis, those whose have endometriosis, those who have had endometriosis, those who at risk of a recurrence of endometriosis, and those who are at risk of developing endometriosis.

In some embodiments, an endometriosis associated miRNA-binding molecule is used in vivo for the diagnosis of endometriosis. In some embodiments, the endometriosis associated miRNA-binding molecule is nucleic acid that hybridizes with an endometriosis associated miRNA of the disclosure.

In some embodiments, the test sample is a sample containing at least a fragment of a nucleic acid comprising a miRNA associated with endometriosis. The term, “fragment,” as used herein, indicates that the portion of a nucleic acid (e.g., DNA, mRNA or cDNA) that is sufficient to identify it as comprising a miRNA associated with endometriosis.

In some embodiments, the test sample is prepared from a biological sample obtained from the subject. The biological sample can be a sample from any source which contains a nucleic acid comprising endometriosis associated miRNA, such as a body fluid (e.g., blood, plasma, serum, saliva, urine, etc.), or a tissue, or an exosome, or a cell, or a combination thereof. A biological sample can be obtained by appropriate methods, such as, by way of examples, biopsy or fluid draw. The biological sample can be used as the test sample; alternatively, the biological sample can be processed to enhance access to polypeptides, nucleic acids, or copies of nucleic acids (e.g., copies of nucleic acids comprising a miRNA associated with endometriosis), and the processed biological sample can then be used as the test sample. For example, in various embodiments, nucleic acid is prepared from a biological sample, for use in the methods. Alternatively, or in addition, an amplification method can be used to amplify nucleic acids comprising all or a fragment of a nucleic acid in a biological sample, for use as the test sample in the assessment of the expression level of a miRNA associated with endometriosis.

The test sample is assessed to determine the level of expression of at least one miRNA associated with endometriosis present in the nucleic acid of the subject. In general, detecting a miRNA may be carried out by determining the presence or absence of a nucleic acid containing a miRNA of interest in the test sample.

In some embodiments, hybridization methods, such as Northern analysis, or in situ hybridizations, can be used (see Current Protocols in Molecular Biology, 2012, Ausubel, F. et al., eds., John Wiley & Sons, including all supplements). For example, the presence of a miRNA associated with endometriosis can be indicated by hybridization to a nucleic acid probe. A “nucleic acid probe,” as used herein, can be a nucleic acid probe, such as a DNA probe or an RNA probe. For representative examples of use of nucleic acid probes, see, for example, U.S. Pat. Nos. 5,288,611 and 4,851,330.

To detect at least one miRNA of interest, a hybridization sample is formed by contacting the test sample with at least one nucleic acid probe. An exemplary probe for detecting miRNA is a labeled nucleic acid probe capable of hybridizing to miRNA. The nucleic acid probe can be, for example, a full-length nucleic acid molecule, or a portion thereof, such as an oligonucleotide of at least 10, 15, or 25 nucleotides in length and sufficient to specifically hybridize under stringent conditions to appropriate miRNA. The hybridization sample is maintained under conditions which are sufficient to allow specific hybridization of the nucleic acid probe to a miRNA target of interest. Specific hybridization can be performed under high stringency conditions or moderate stringency conditions, as appropriate. In some embodiments, the hybridization conditions for specific hybridization are high stringency. Specific hybridization, if present, is then detected using standard methods. If specific hybridization occurs between the nucleic acid probe and a miRNA in the test sample, the sequence that is present in the nucleic acid probe is also present in the miRNA of the subject. More than one nucleic acid probe can also be used concurrently in this method. Specific hybridization of any one of the nucleic acid probes is indicative of the presence of the miRNA of interest, as described herein.

Alternatively, a peptide nucleic acid (PNA) probe can be used instead of a nucleic acid probe in the hybridization methods described herein. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, 1994, Nielsen et al., Bioconjugate Chemistry 5:1). The PNA probe can be designed to specifically hybridize to a nucleic acid sequence comprising at least one miRNA of interest. Hybridization of the PNA probe to a nucleic acid sequence is indicative of the presence of a miRNA of interest.

Direct sequence analysis can also be used to detect miRNAs of interest. A sample comprising nucleic acid can be used, and PCR or other appropriate methods can be used to amplify all or a fragment of the nucleic acid, and/or its flanking sequences.

In some embodiments, arrays of oligonucleotide probes that are complementary to target nucleic acid sequences from a subject can be used to detect, identify and quantify miRNAs associated with endometriosis. For example, in some embodiments, an oligonucleotide array can be used. Oligonucleotide arrays typically comprise a plurality of different oligonucleotide probes that are coupled to a surface of a substrate in different pre-defined or addressed locations. These oligonucleotide arrays, also referred to as “Genechips,” have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods. See Fodor et al., Science, 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186. Techniques for the synthesis of these arrays using mechanical synthesis methods are described in, e.g., U.S. Pat. No. 5,384,261.

After an oligonucleotide array is prepared, a sample containing miRNA is hybridized with the array and scanned for miRNAs. Hybridization and scanning are generally carried out by methods described herein and also in, e.g., Published PCT Application Nos. WO 92/10092 and WO 95/11995, and U.S. Pat. No. 5,424,186, the entire teachings of which are incorporated by reference herein.

In brief, a target miRNA sequence is amplified by suitable amplification techniques, e.g., RT, PCR. Typically, this involves the use of primer sequences that are complementary to the target miRNA. Amplified target, generally incorporating a label, is then hybridized with the array under appropriate conditions. Upon completion of hybridization and washing of the array, the array is scanned to determine the position on the array to which the target sequence hybridizes. The hybridization data obtained from the scan is typically in the form of fluorescence intensities as a function of location on the array.

Other methods of nucleic acid analysis can be used to detect miRNAs of interest. Representative methods include direct manual sequencing (1988, Church and Gilbert, Proc. Natl. Acad. Sci. USA 81:1991-1995; 1977, Sanger et al., Proc. Natl. Acad. Sci. 74:5463-5467; Beavis et al. U.S. Pat. No. 5,288,644); automated fluorescent sequencing; single-stranded conformation polymorphism assays (SSCP); clamped denaturing gel electrophoresis (CDGE); denaturing gradient gel electrophoresis (DGGE) (Sheffield et al., 1981, Proc. Natl. Acad. Sci. USA 86:232-236), mobility shift analysis (Orita et al., 1989, Proc. Natl. Acad. Sci. USA 86:2766-2770; Rosenbaum and Reissner, 1987, Biophys. Chem. 265:1275; 1991, Keen et al., Trends Genet. 7:5); RNase protection assays (Myers, et al., 1985, Science 230:1242); Luminex xMAP™ technology; high-throughput sequencing (HTS) (Gundry and Vijg, 2011, Mutat Res, doi: 10.1016/j.mrfmmm.2011.10.001); next-generation sequencing (NGS) (Voelkerding et al., 2009, Clinical Chemistry 55:641-658; Su et al., 2011, Expert Rev Mol Diagn. 11:333-343; Ji and Myllykangas, 2011, Biotechnol Genet Eng Rev 27:135-158); and/or ion semiconductor sequencing (Rusk, 2011, Nature Methods doi:10.1038/nmeth.f.330; Rothberg et al., 2011, Nature 475:348-352). These and other methods, alone or in combination, can be used to detect and quantity of at least one miRNA of interest, in a biological sample obtained from a subject. In some embodiments of the disclosure, the methods of assessing a biological sample to detect and quantify a miRNA of interest, as described herein, are used to diagnose, assess and characterize endometriosis in a subject in need thereof.

In some embodiments, sequencing can be performed using a next generation sequencing assay. As used herein, the term “next generation” is well-understood in the art and generally refers to any high-throughput sequencing approach including, but not limited to one or more of the following: massively-parallel signature sequencing, pyrosequencing (e.g., using a Roche 454 sequencing device), Illumina (Solexa) sequencing, sequencing by synthesis (Illumina), Ion torrent sequencing, sequencing by ligation (e.g., SOLiD sequencing), single molecule real-time (SMRT) sequencing (e.g., Pacific Bioscience), polony sequencing, DNA nanoball sequencing, heliscope single molecule sequencing (Helicos Biosciences), and nanopore sequencing (e.g., Oxford Nanopore). In some cases, the sequencing assay uses nanopore sequencing. In some cases, the sequencing assay includes some form of Sanger sequencing. In some cases, the sequencing involves shotgun sequencing; in some cases, the sequencing includes bridge PCR. In some cases, the sequencing is broad spectrum. In some cases, the sequencing is targeted.

The probes and primers according to the disclosure can be labeled directly or indirectly with a radioactive or nonradioactive compound in order to obtain a detectable and/or quantifiable signal; the labeling of the primers or of the probes according to the disclosure is carried out with radioactive elements or with nonradioactive molecules. Among the radioactive isotopes used, mention may be made of ³²P, ³³P, ³⁵S or ³H. The nonradioactive entities are selected from ligands such as biotin, avidin, streptavidin or digoxigenin, haptenes, dyes, and luminescent agents such as radioluminescent, chemoluminescent, bioluminescent, fluorescent or phosphorescent agents.

Nucleic acids can be obtained from the biological sample using suitable techniques. Nucleic acid herein includes RNA, including mRNA, miRNA, etc. The nucleic acid can be double-stranded or single-stranded (i.e., a sense or an antisense single strand) and can be complementary to a nucleic acid encoding a polypeptide. The nucleic acid content may also be obtained from an extraction performed on a fresh or fixed biological sample.

There are many suitable methods for the detection of specific nucleic acid sequences and new methods are continually reported. One such category of methods involves specific hybridization reactions as detailed below.

In the Northern blot, the nucleic acid probe is preferably labeled with a tag. That tag can be a radioactive isotope, a fluorescent dye or another conveniently detectable moiety. Another type of process for the specific detection of nucleic acids of exogenous organisms in a body sample are the hybridization methods as exemplified by U.S. Pat. Nos. 6,159,693 and 6,270,974, and related patents. To briefly summarize one of those methods, a nucleic acid probe of at least 10 nucleotides, preferably at least 15 nucleotides, more preferably at least 25 nucleotides, having a sequence complementary to a region of the target nucleic acid of interest is hybridized in a sample, subjected to depolymerizing conditions, and the sample is treated with an ATP/luciferase system, which will luminesce if the nucleic sequence is present.

In quantitative Northern blotting, levels of the polymorphic nucleic acid can be compared to wild-type levels of the nucleic acid.

A further process for the detection of hybridized nucleic acid takes advantage of the polymerase chain reaction (PCR). The PCR process is described in e.g. U.S. Pat. Nos. 4,683,195, 4,683,202, and 4,800,159. To briefly summarize PCR, nucleic acid primers, complementary to opposite strands of a nucleic acid amplification target nucleic acid sequence, are permitted to anneal to the denatured sample. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product.

In PCR, the nucleic acid probe can be labeled with a tag as discussed before. Most preferably the detection of the duplex is done using at least one primer directed to the target nucleic acid. In some embodiments of PCR, the detection of the hybridized duplex comprises electrophoretic gel separation followed by dye-based visualization.

Nucleic acid amplification procedures by PCR are described in e.g. U.S. Pat. No. 4,683,202. Briefly, the primers anneal to the target nucleic acid at sites distinct from one another and in an opposite orientation. A primer annealed to the target sequence is extended by the enzymatic action of a heat stable polymerase. The extension product is then denatured from the target sequence by heating, and the process is repeated. Successive cycling of this procedure on both strands provides exponential amplification of the region flanked by the primers.

Amplification is then performed using a PCR-type technique, that is to say the PCR technique or any other related technique. Two primers, complementary to the target nucleic acid sequence are then added to the nucleic acid content along with a polymerase, and the polymerase amplifies the DNA region between the primers.

Amplification may refer to any method for increasing the number of copies of a nucleic acid sequence. For example, the amplification may be performed with a polymerase, e.g., in one or more polymerase chain reactions. Amplification may be performed using other suitable methods. These methods often depend on the product catalyzed formation of multiple copies of a nucleic acid or its complement. One of such methods is polymerase chain reaction (PCR), including AFLP (amplified fragment length polymorphism) PCR, allele-specific PCR, Alu PCR, assembly, asymmetric PCR, colony PCR, helicase dependent PCR, hot start PCR, inverse PCR, in situ PCR, intersequence-specific PCR or IS SR PCR, digital PCR, droplet digital PCR, linear-after-the-exponential-PCR or Late PCR, long PCR, nested PCR, real-time PCR, duplex PCR, multiplex PCR, quantitative PCR, or single cell PCR. Other amplification methods may also be used, including ligase chain reaction (LCR), nucleic acid sequence-based amplification (NASBA), linear amplification, isothermal linear amplification, Q-beta-replicase method, 3SR, Transcription Mediated Amplification (TMA), Strand Displacement Amplification (SDA), or Rolling Circle Amplification (RCA).

Stem-loop RT-PCR is a PCR method that is useful in the methods of the disclosure to amplify and quantify miRNAs of interest (See Caifu et al., 2005, Nucleic Acids Research 33:e179; Mestdagh et al., 2008, Nucleic Acids Research 36:e143; Varkonyi-Gasic et al., 2011, Methods Mol Biol. 744:145-57). Briefly, the method includes two steps: RT and real-time PCR. First, a stem-loop RT primer is hybridized to a miRNA molecule and then reverse transcribed with a reverse transcriptase. Then, the RT products are quantified using conventional real-time PCR.

The expression specifically hybridizing in stringent conditions refers to a hybridizing step in the process of the disclosure where the oligonucleotide sequences selected as probes or primers are of adequate length and sufficiently unambiguous so as to minimize the amount of non-specific binding that may occur during the amplification. The oligonucleotide probes or primers herein described may be prepared by any suitable methods such as chemical synthesis methods.

Hybridization is typically accomplished by annealing the oligonucleotide probe or primer to the template nucleic acid under conditions of stringency that prevent non-specific binding but permit binding of this template nucleic acid which has a significant level of homology with the probe or primer.

Among the conditions of stringency is the melting temperature (Tm) for the amplification step using the set of primers, which is in the range of about 50° C. to about 95° C. Typical hybridization and washing stringency conditions depend in part on the size (i.e., number of nucleotides in length) of the template nucleic acid or the oligonucleotide probe, the base composition and monovalent and divalent cation concentrations (Ausubel et al., 1994, eds Current Protocols in Molecular Biology).

In some embodiments, the process for determining the quantitative and qualitative profile according to the present disclosure is characterized in that the amplifications are real-time amplifications performed using a labeled probe, preferably a labeled hydrolysis-probe, capable of specifically hybridizing in stringent conditions with a segment of a nucleic acid sequence, or polymorphic nucleic acid sequence. The labeled probe is capable of emitting a detectable signal every time each amplification cycle occurs.

Real-time amplification, such as real-time PCR, can be performed in various different iterations; it can be performed e.g. using various categories of probes, such as hydrolysis probes, hybridization adjacent probes, or molecular beacons. The techniques employing hydrolysis probes or molecular beacons are based on the use of a fluorescence quencher/reporter system, and the hybridization adjacent probes are based on the use of fluorescence acceptor/donor molecules.

Hydrolysis probes with a fluorescence quencher/reporter system are available in the market and are for example commercialized by the Applied Biosystems group (USA). Many fluorescent dyes may be employed, such as FAM dyes (6-carboxy-fluorescein), or any other dye phosphoramidite reagents.

Among the stringent conditions applied for any one of the hydrolysis-probes of the present disclosure is the Tm, which is in the range of about 50° C. to 95° C. Preferably, the Tm for any one of the hydrolysis-probes of the present disclosure is in the range of about 55° C. to about 80° C. Most preferably, the Tm applied for any one of the hydrolysis-probes of the present disclosure is about 75° C.

In some embodiments, the process for determining the quantitative and qualitative profile according to the present disclosure is characterized in that the amplification products can be elongated, wherein the elongation products are separated relative to their length. The signal obtained for the elongation products is measured, and the quantitative and qualitative profile of the labeling intensity relative to the elongation product length is established.

The elongation step, also called a run-off reaction, allows one to determine the length of the amplification product. The length can be determined using conventional techniques, for example, using gels such as polyacrylamide gels for the separation, DNA sequencers, and adapted software. Because some mutations display length heterogeneity, some mutations can be determined by a change in length of elongation products.

In one aspect, the disclosure includes a primer that is complementary to a nucleic acid sequence of the miRNA of interest, and more particularly the primer includes 12 or more contiguous nucleotides substantially complementary to the sequence of the miRNA of interest. Preferably, a primer featured in the disclosure includes a nucleotide sequence sufficiently complementary to hybridize to a nucleic acid sequence of about 12 to 25 nucleotides. More preferably, the primer differs by no more than 1, 2, or 3 nucleotides from the target nucleotide sequence. In another aspect, the length of the primer can vary in length, preferably about 15 to 28 nucleotides in length (e.g., 15, 16, 17, 18, 19, 20, 21, 22, 23, 24, 25, 26, 27 or 28 nucleotides in length).

### Quantitative Algorithm

In some embodiments, the method comprises using a quantitative algorithm to determine if the expression level of a set of biomarkers in the biological sample is statistically different than the expression level in a control sample. The algorithm may be a trained algorithm. In various embodiments, the algorithm is drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; a plurality of algorithms arranged in a committee network; and forward floating search or backward floating search algorithms. Such algorithms may be used in supervised or unsupervised learning modes. In various embodiments, quantitative algorithms according to the disclosure can be used to determine the extent, severity, or stage of disease, to determine the right treatment approach (e.g., oral contraceptives, disease-specific therapy, surgical intervention), to select the appropriate dose for a medical treatment, to determine whether a patient is likely to respond to a particular medical or surgical treatment, to monitor response to treatment, or to monitor disease progression.

In some embodiments, the methods according to the disclosure include deriving a numerical value, index or score from the quantitative algorithm or mathematical formula. In some embodiments, the derived numerical value can serve as a cut off value for distinguishing between two or more potential outcomes (e.g., high or low risk of disease presence, progression or recurrence or stage of disease.) In some embodiments, a derived numerical value serves as a cutoff value for an expression level of at least one miRNA. In some embodiments, a derived numerical value serves as a cutoff value for an expression level of at least one miRNA in order determine the extent, severity, or stage of disease. In some embodiments, a derived numerical value serves as a cutoff value for an expression level of at least one miRNA in order to determine the right treatment approach (e.g., oral contraceptives, disease-specific therapy, surgical intervention). In some embodiments, a derived numerical value serves as a cutoff value for an expression level of at least one miRNA in order to select the appropriate dose for a medical treatment.) In some embodiments, a derived numerical value serves as a cutoff value for an expression level of at least one miRNA in order to determine whether a patient is likely to respond to a particular medical or surgical treatment. In some embodiments, a derived numerical value serves as a cutoff value for an expression level of at least one miRNA in order to monitor response to treatment. In some embodiments, a derived numerical value serves as a cutoff value for an expression level of at least one miRNA in order to monitor disease progression.

### Determining Effectiveness of Therapy or Prognosis

In one aspect, the level of one or more circulating miRNAs in a biological sample of a patient is used to monitor the effectiveness of treatment or the prognosis of disease. In some embodiments, the level of one or more circulating miRNAs in a test sample obtained from a treated patient can be compared to the level from a reference sample obtained from that patient before initiation of a treatment. Clinical monitoring of treatment typically entails that each patient serve as his or her own baseline control. In some embodiments, test samples are obtained at multiple time points following administration of the treatment. In these embodiments, measurement of level of one or more circulating miRNAs in the test samples provides an indication of the extent and duration of in vivo effect of the treatment.

Measurement of biomarker levels allow for the course of treatment of a disease to be monitored. The effectiveness of a treatment regimen for a disease can be monitored by detecting one or more biomarkers in an effective amount from samples obtained from a subject over time and comparing the amount of biomarkers detected. For example, a first sample can be obtained before the subject receives treatment and one or more subsequent samples are taken after or during treatment of the subject. Changes in biomarker levels across the samples may provide an indication as to the effectiveness of the therapy.

In some embodiments, the disclosure provides a method for monitoring the levels of miRNAs in response to treatment. For example, in certain embodiments, the disclosure provides for a method of determining the efficacy of treatment in a subject, by measuring the levels of one or more miRNAs described herein. In some embodiments, the level of the one or more miRNAs can be measured over time, where the level at one timepoint after the initiation of treatment is compared to the level at another timepoint after the initiation of treatment. In some embodiments, the level of the one or more miRNAs can be measured over time, where the level at one timepoint after the initiation of treatment is compared to the level before initiation of treatment. In some embodiments, the disclosure provides a method for monitoring at least one of miR-125, miR-150, miR-342, miR-145, miR-143, miR-500, miR-451, miR-18, miR-214, miR-126, miR-6755, miR-3613, miR-553, miR-4668, let-7b, miR-135, miR-449a, miR-34c, miR-200a, miR-200b, miR-141, miR-125b-5p, miR-150-5p, miR-342-3p, miR-145-5p, miR-143-3p, miR-500a-3p, miR-451a, miR-18a-5p, miR-6755-3p, and miR-3613-5p after treatment.

In some embodiments, the disclosure provides a method for assessing the efficacy of an endometriosis treatment. For example, in some embodiments, the method indicates that the treatment is effective when the level of at least one of miR-125, miR-150, miR-342, miR-145, miR-143, miR-500, miR-451, miR-18, miR-214, miR-126, miR-6755, miR-3613, miR-553, miR-4668, let-7b, miR-135, miR-449a, miR-34c, miR-200a, miR-200b, miR-141, miR-125b-5p, miR-150-5p, miR-342-3p, miR-145-5p, miR-143-3p, miR-500a-3p, miR-451a, miR-18a-5p, miR-6755-3p, and miR-3613-5p is decreased in a sample of a treated subject as compared to a control diseased subject or population not receiving treatment. In some embodiments, the method indicates that the treatment is effective when the level of at least one of miR-125, miR-150, miR-342, and miR-451 is decreased in a sample of a treated subject as compared to a control diseased subject or population not receiving treatment.

In some embodiments, the method indicates that the treatment is effective when the level of at least one of miR-125, miR-150, miR-342, miR-145, miR-143, miR-500, miR-451, miR-18, miR-214, miR-126, miR-6755, miR-3613, miR-553, miR-4668, let-7b, miR-135, miR-449a, miR-34c, miR-200a, miR-200b, miR-141, miR-125b-5p, miR-150-5p, miR-342-3p, miR-145-5p, miR-143-3p, miR-500a-3p, miR-451a, miR-18a-5p, miR-6755-3p, and miR-3613-5p is increased in a sample of a treated subject as compared to a control diseased subject or population not receiving treatment. In some embodiments, the method indicates that the treatment is effective when the level of at least one of let 7b and miR-3613 is increased in a sample of a treated subject as compared to a control diseased subject or population not receiving treatment.

In some embodiments, the disclosure provides a method for assessing the efficacy of an endometriosis treatment. For example, in some embodiments, the method indicates that the treatment is effective when the level of miR-125b-5p is decreased in a sample of a treated subject as compared to a control diseased subject or population not receiving treatment. In some embodiments, the method indicates that the treatment is effective when the level of miR-150-5p is decreased in a sample of a treated subject as compared to a control diseased subject or population not receiving treatment. In some embodiments, the method indicates that the treatment is effective when the level of miR-3613-5p is increased in a sample of a treated subject as compared to a control diseased subject or population not receiving treatment.

To identify therapeutics or drugs that are appropriate for a specific subject, a test sample from the subject can also be exposed to a therapeutic agent or a drug, and the level of one or more biomarkers can be determined. Biomarker levels can be compared to a sample derived from the subject before and after treatment or exposure to a therapeutic agent or a drug or can be compared to samples derived from one or more subjects who have shown improvements relative to a disease as a result of such treatment or exposure. Thus, in one aspect, the disclosure provides a method of assessing the efficacy of a therapy with respect to a subject comprising taking a first measurement of a biomarker panel in a first sample from the subject; effecting the therapy with respect to the subject; taking a second measurement of the biomarker panel in a second sample from the subject and comparing the first and second measurements to assess the efficacy of the therapy.

Additionally, therapeutic agents suitable for administration to a particular subject can be identified by detecting one or more biomarkers in an effective amount from a sample obtained from a subject and exposing the subject-derived sample to a test compound that determines the amount of the biomarker(s) in the subject-derived sample. Accordingly, treatments or therapeutic regimens for use in subjects having endometriosis can be selected based on the amounts of biomarkers in samples obtained from the subjects and compared to a reference value. Two or more treatments or therapeutic regimens can be evaluated in parallel to determine which treatment or therapeutic regimen would be the most efficacious for use in a subject to delay onset, or slow progression of a disease. In various embodiments, a recommendation is made on whether to initiate or continue treatment of a disease.

A prognosis may be expressed as the amount of time a patient can be expected to survive. Alternatively, a prognosis may refer to the likelihood that the disease goes into remission or to the amount of time the disease can be expected to remain in remission. Prognosis can be expressed in various ways; for example, prognosis can be expressed as a percent chance that a patient will survive after one year, five years, ten years or the like. Alternatively, prognosis may be expressed as the number of years, on average that a patient can expect to survive as a result of a condition or disease. The prognosis of a patient may be considered as an expression of relativism, with many factors affecting the ultimate outcome. For example, for patients with certain conditions, prognosis can be appropriately expressed as the likelihood that a condition may be treatable or curable, or the likelihood that a disease will go into remission, whereas for patients with more severe conditions, prognosis may be more appropriately expressed as likelihood of survival for a specified period of time. Additionally, a change in a clinical factor from a baseline level may impact a patient's prognosis, and the degree of change in level of the clinical factor may be related to the severity of adverse events. Statistical significance is often determined by comparing two or more populations and determining a confidence interval and/or a p value.

Multiple determinations of circulating miRNA levels can be made, and a temporal change in activity can be used to determine a prognosis. For example, comparative measurements are made of the circulating miRNA of an acellular body fluid in a patient at multiple time points, and a comparison of a circulating miRNA value at two or more time points may be indicative of a particular prognosis.

In certain embodiments, the levels of activity of one or more circulating miRNAs are used as indicators of an unfavorable prognosis. According to the current disclosure, the determination of prognosis can be performed by comparing the measured circulating miRNA level to levels determined in comparable samples from healthy individuals or to levels corresponding with favorable or unfavorable outcomes. The circulating miRNA levels obtained may depend on a number of factors, including, but not limited to, the laboratory performing the assays, the assay methods used, the type of body fluid sample used and the type of disease a patient is afflicted with. According to the method, values can be collected from a series of patients with a particular disorder to determine appropriate reference ranges of circulating miRNA for that disorder. Various techniques are available for performing a retrospective study that compares the determined levels to the observed outcome of the patients and establishing ranges of levels that can be used to designate the prognosis of the patients with a particular disorder. For example, levels in the lowest range would be indicative of a more favorable prognosis, while circulating miRNA levels in the highest ranges would be indicative of an unfavorable prognosis. Thus, in this aspect the term “elevated levels” refers to levels of that are above the range of the reference value. In some embodiments patients with “high” or “elevated” levels have levels that are higher than the median activity in a population of patients with that disease. In certain embodiments, “high” or “elevated” levels for a patient with a particular disease refers to levels that are above the median values for patients with that disorder and are in the upper 40% of patients with the disorder, or to levels that are in the upper 20% of patients with the disorder, or to levels that are in the upper 10% of patients with the disorder, or to levels that are in the upper 5% of patients with the disorder. Because the level of circulating miRNA in a test sample from a patient relates to the prognosis of a patient in a continuous fashion, the determination of prognosis can be performed using statistical analyses to relate the determined circulating miRNA levels to the prognosis of the patient. A skilled artisan is capable of designing appropriate statistical methods. For example, the methods may employ the chi-squared test, the Kaplan-Meier method, the log-rank test, multivariate logistic regression analysis, Cox's proportional-hazard model and the like in determining the prognosis. Computers and computer software programs may be used in organizing data and performing statistical analyses. The approach by Giles et. al., British Journal of Hemotology, 121:578-585, is exemplary. As in Giles et al., associations between categorical variables (e.g., miRNA levels and clinical characteristics) can be assessed via cross-tabulation and Fisher's exact test. Unadjusted survival probabilities can be estimated using the method of Kaplan and Meier. The Cox proportional hazards regression model also can be used to assess the ability of patient characteristics (such as miRNA levels) to predict survival, with ‘goodness of fit’ assessed by the Grambsch-Therneau test, Schoenfeld residual plots, martingale residual plots and likelihood ratio statistics (see Grambsch et al, 1995). In some embodiments, this approach can be adapted as a simple computer program that can be used with personal computers or personal digital assistants (PDA). The prediction of patients' survival time in based on their circulating miRNA levels can be performed via the use of a visual basic for applications (VBA) computer program developed within Microsoft Excel. The core construction and analysis may be based on the Cox proportional hazard models. The VBA application can be developed by obtaining a base hazard rate and parameter estimates. These statistical analyses can be performed using a statistical program such as the SAS proportional hazards regression, PHREG, procedure. Estimates can then be used to obtain probabilities of surviving from one to 24 months given the patient's covariates. The program can make use of estimated probabilities to create a graphical representation of a given patient's predicted survival curve. In certain embodiments, the program also provides 6-month, 1-year and 18-month survival probabilities. A graphical interface can be used to input patient characteristics in a user-friendly manner. In some embodiments of the disclosure, multiple prognostic factors, including circulating miRNA level, are considered when determining the prognosis of a patient. For example, the prognosis of an endometriosis subject or may be determined based on the presence of miRNA in a body fluid and one or more prognostic factors selected from the group consisting of cytogenetics, performance status, age, gender and contemporary diagnosis. In another example, the prognosis of a cancer patient may be determined based on circulating miRNA and one or more prognostic factors selected from the group consisting of cytogenetics, performance status, age, gender and contemporary diagnosis. In certain embodiments, other prognostic factors may be combined with the circulating miRNA level or other biomarkers in the algorithm to determine prognosis with greater accuracy.

### Treatments

In one aspect, the disclosure provides a method of treating or preventing endometriosis or a disease or disorder associated with endometriosis in a subject. For example, in certain embodiments, the subject has been identified as having a differentially expressed level of one or more of miR-125, miR-150, miR-342, miR-145, miR-143, miR-500, miR-451, miR-18, miR-214, miR-126, miR-6755, miR-3613, miR-553, miR-4668, let-7b, miR-135, miR-449a, miR-34c, miR-200a, miR-200b, miR-141, miR-125b-5p, miR-150-5p, miR-342-3p, miR-145-5p, miR-143-3p, miR-500a-3p, miR-451a, miR-18a-5p, miR-6755-3p, or miR-3613-5p.

In some embodiments, the method comprises administering to the subject an effective amount of a pharmaceutical agent for the treatment of endometriosis. Exemplary pharmaceutical agents for the treatment of endometriosis include, but are not limited to, pain medications (e.g., nonsteroidal anti-inflammatory drugs (NSAIDs), ibuprofen (Advil, Motrin IB, others) and naproxen sodium (Aleve)), hormone therapy (e.g., birth control pills, patches, intrauterine devices and vaginal rings, gonadotropin-releasing hormone (Gn-RH) agonists and antagonists, progestin therapy, contraceptive injection, and aromatase inhibitors.

In some embodiments, the method comprises administering to the subject surgical treatment, for example, laparoscopic surgery to remove the endometriosis, hysterectomy or oophoprectomy. Subjects to which administration of the compositions according to the disclosure is contemplated include, but are not limited to, humans and other primates, mammals including commercially relevant mammals such as non-human primates, cattle, pigs, horses, sheep, cats, and dogs.

Pharmaceutical compositions according to the present disclosure may be administered in a manner appropriate to the disease to be treated (or prevented). The quantity and frequency of administration will be determined by such factors as the condition of the subject, and the type and severity of the subject's disease, although appropriate dosages may be determined by clinical trials.

When “therapeutic amount” is indicated, the precise amount of the compositions of the present disclosure to be administered can be determined by a physician with consideration of individual differences in age, weight, disease type, extent of disease, and condition of the patient (subject).

Typically, dosages of a compound according to the disclosure which may be administered to an animal range in amount from about 0.01 mg to 20 about 100 g per kilogram of body weight of the animal. While the precise dosage administered will vary depending upon any number of factors, including, but not limited to, the type of animal and type of disease state being treated, the age of the animal and the route of administration. In some embodiments, the dosage of the compound will vary from about 1 mg to about 100 mg per kilogram of body weight of the animal. In some embodiments, the dosage will vary from about 1 μg to about 1 g per kilogram of body weight of the animal. The compound can be administered to an animal as frequently as several times daily, or it can be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

### Systems for Diagnosing and Guiding Treatment of Endometriosis

The present disclosure also provides for software for guiding the diagnosis and treatment of endometriosis. The software combines one or more of the methods described elsewhere herein to diagnose or guide treatment of endometriosis.

In various embodiments, the present disclosure includes software executing instructions and algorithms relating to the methods provided herein. Such software may be stored on a non-transitory computer-readable medium, wherein the software performs some or all of the steps according to the present disclosure when executed on a processor.

Aspects of the disclosure relate to algorithms of the disclosure executed in computer software. Though certain embodiments may be described as written in particular programming languages, or executed on particular operating systems or computing platforms, it is understood that the systems and methods of the present disclosure are not limited to any particular computing language, platform, or combination thereof. Software executing the algorithms described herein may be written in any programming language compiled or interpreted, including but not limited to C, C++, C#, Objective-C, Java, JavaScript, Python, PHP, Perl, Ruby, or Visual Basic. It is further understood that elements of the present disclosure may be executed on any acceptable computing platform, including but not limited to a server, a cloud instance, a workstation, a thin client, a mobile device, an embedded microcontroller, a television, or any other suitable computing device.

Parts of this disclosure provide for software running on a computing device. Though software described herein may be disclosed as operating on one particular computing device (e.g., a dedicated server or a workstation), it is understood in the art that software is intrinsically portable and that most software running on a dedicated server may also be run, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device.

Similarly, embodiments according to the present disclosure are described as communicating over a variety of wireless or wired computer networks. As used herein, the words “network”, “networked”, and “networking” are understood to encompass wired Ethernet, fiber optic connections, wireless connections including any of the various 802.11 standards, cellular WAN infrastructures such as 3G or 4G/LTE networks, Bluetooth®, Bluetooth® Low Energy (BLE) or Zigbee® communication links, or any other method by which one electronic device is capable of communicating with another. In some embodiments, elements of the networked portion of embodiments according to the present disclosure may be implemented over a Virtual Private Network (VPN).

### Kits

The present disclosure also pertains to kits useful in the methods of the disclosure. Such kits comprise components useful in any of the methods described herein, including for example, hybridization probes or primers (e.g., labeled probes or primers), reagents for detection of labeled molecules, oligonucleotide arrays, restriction enzymes, antibodies, allele-specific oligonucleotides, materials and reagents for amplification of a subject's nucleic acids, materials and reagents for reverse transcribing a subject's RNA, materials and reagents for analyzing a subject's nucleic acid sequence, and instructional materials. For example, in some embodiments, the kit comprises components useful for the detection and quantification of at least one miRNA associated with endometriosis. In some embodiments of the disclosure, the kit comprises components for detecting one or more of the miRNAs associated with endometriosis as elsewhere described herein.

The present disclosure also provides kits for diagnosing endometriosis or reduced fertility caused by endometriosis, comprising a probe for one or more nucleic acid biomarkers differentially expressed in endometriosis. In one particular embodiment, the kit comprises reagents for quantitative amplification of the selected biomarkers. Alternatively, the kit may comprise a microarray. In some embodiments the kit comprises 2 or more probes. In some embodiments, the kits may contain 3, 4, 5, 6, 7, 8, 9, 10, 15, 20, 25, or more probes.

The present disclosure also pertains to kits useful in the methods of the disclosure. Such kits comprise various combinations of components useful in any of the methods described elsewhere herein, including for example, materials for quantitatively analyzing a biomarker of the disclosure (e.g., at least one miR), materials for assessing the activity of a biomarker of the disclosure (e.g., at least one miR), and instructional material. For example, in some embodiments, the kit comprises components useful for the quantification of a nucleic acid according to the disclosure in a biological sample.

In a further embodiment, the kit comprises the components of an assay for monitoring the effectiveness of a treatment administered to a subject in need thereof, containing instructional material and the components for determining whether the level of a biomarker of the disclosure in a biological sample obtained from the subject is modulated during or after administration of the treatment. In various embodiments, to determine whether the level of a biomarker of the disclosure is modulated in a biological sample obtained from the subject, the level of the biomarker is compared with the level of at least one comparator control contained in the kit, such as a positive control, a negative control, a historical control, a historical norm, or the level of another reference molecule in the biological sample. In certain embodiments, the ratio of the biomarker and a reference molecule is determined to aid in the monitoring of the treatment.

### EXPERIMENTAL EXAMPLES

The invention can be further understood in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein. Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Serum microRNAs Used to Diagnose Endometriosis Before Surgical Diagnosis: A Prospective Study

A prospective clinical evaluation was performed to assess the validity of a panel of miRNA markers to function as markers of endometriosis within a diverse population. This larger patient population comprised women from a variety of ethnic backgrounds, and with all stages of the disease, from mild to severe. Samples were obtained pre-operatively, before surgical diagnosis, and quantitative PCR was used to compare the expression of key miRNAs.

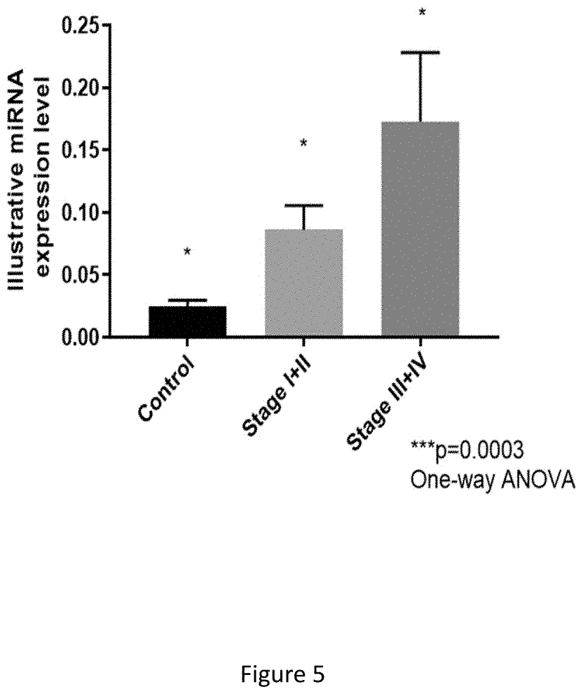

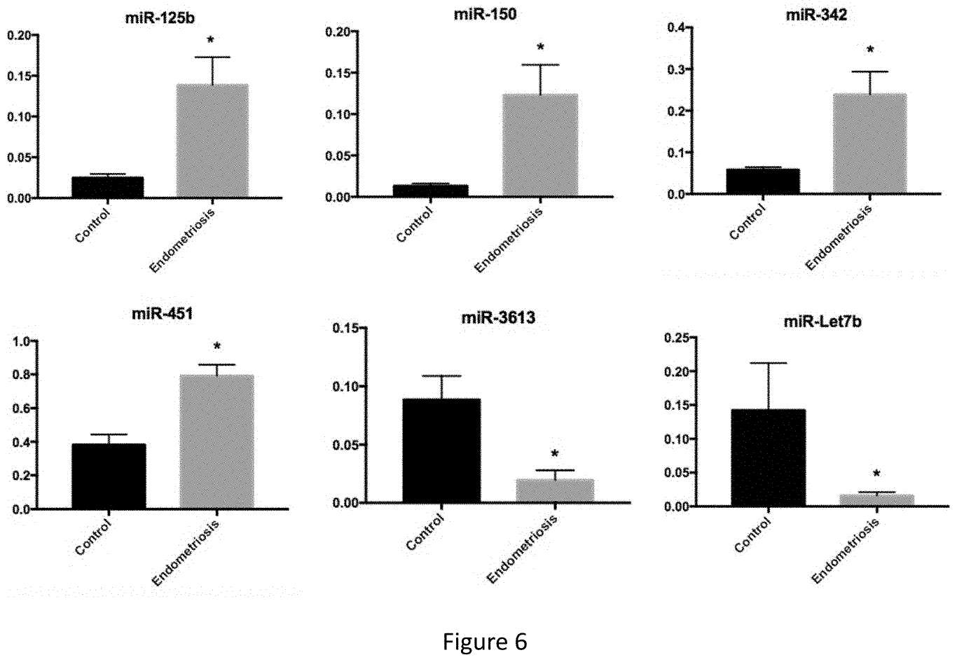

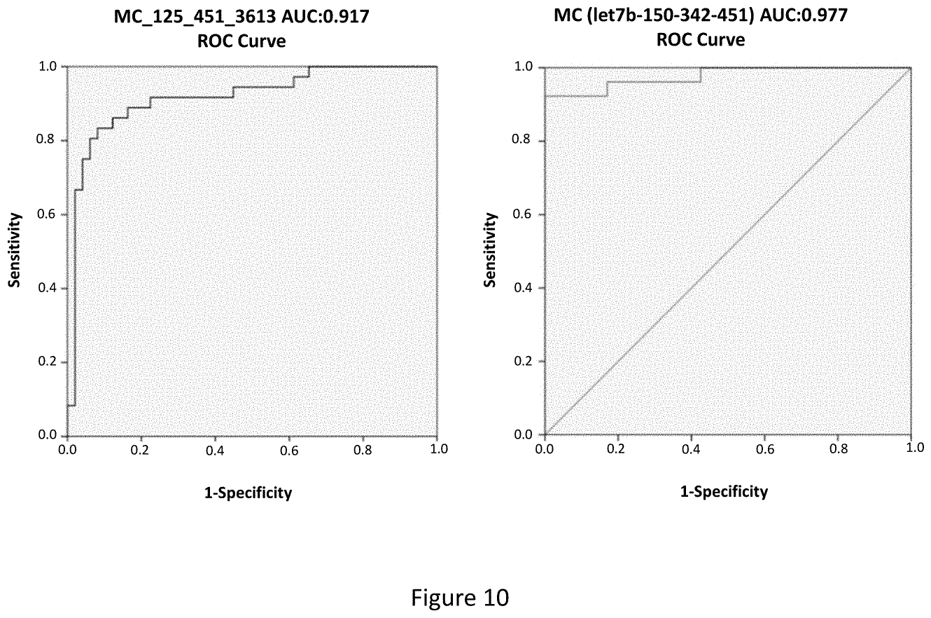

A combination of three miRNAs, miR-125b, miR-451a, and miR-3613, yielded an AUC score of 0.92 when tested against an initial population in this prospective study. Using logistic regression analysis on this prospective study dataset of 86 patients, a new formula combining 4 miRNAs was developed, which had an AUC score=0.977 to diagnose the presence of endometriosis (FIG. 10). In addition, levels of certain miRNAs correlated with the severity of disease, with lower levels in minimal/mild cases, and higher levels in moderate/severe cases (FIG. 5).

Subjects

86 patients were enrolled in the study and analyzed. No significant difference was apparent for ages between the two groups, while a slight difference in BMI (p=0.044) and race composition was observable, consistent with data about prevalence of endometriosis in certain demographics—such as higher prevalence in subjects with lower BMI and individuals of Asian race.

TABLE 1
Study Subject Demographics

| | Control | | Endometriosis | |
|---|---|---|---|---|
| Age (mean, range) | 36.8 | (19-59) | 34.22 | (20-47) |
| BMI (mean, range) | 30.7 | (18.8-47.7) | 28.2 | (18.6-49.6) |
| Race (%) | | | | |

| | | |
|---|---|---|
| Caucasian | 42 | 66.6 |
| Black/African American | 34 | 8.3 |
| Hispanic | 22 | 19.4 |
| Asian | 0 | 5.5 |
| Other | 2 | 0 |

Of the 86 patients enrolled, 36 were subjects with endometriosis, of which 18 were on hormonal medications, and 18 were not undergoing hormonal treatment. The hormonal medications were: combined oral contraceptive pills (OCPs, n=8; 44%), GnRH Agonist (n=6; 33%), Aromatase Inhibitor (n=2; 11%), and Progesterone (n=2; 11%)

Of the 50 control subjects, 14 had no abnormal pathology, 2 had paratubal cysts (4%), 3 had dermoids (6%), 3 had chronic infection (6%), 4 had cystadenoma (8%), and 24 had fibroids (48%).

### Results

Qt-PCR was used to compare expression of key microRNAs, and was consistent with our understanding of miRNAs in endometriosis; there was significantly increased expression of miR-125, miR-150, miR-342, and miR-451 in patients with endometriosis, and significantly reduced expression of miR-3613 and let-7b. The fold changes for a subset of identified miRNAs are provided in Table 2.

TABLE 2
miRNA Fold Changes

| miRNA | Fold change |
|---|---|
| miR-125 | 5.56-fold increase in endometriosis as compared |
| | to control |
| miR-150 | 9.27-fold increase in endometriosis as compared |
| | to control |
| miR-342 | 4.46-fold increase in endometriosis as compared |
| | to control |
| miR-451 | 2.07-fold increase in endometriosis as compared |
| | to control |
| let-7b | 8.89-fold decrease in endometriosis as compared |
| | to control |
| miR-3613 | 4.56-fold decrease in endometriosis as compared |
| | to control |

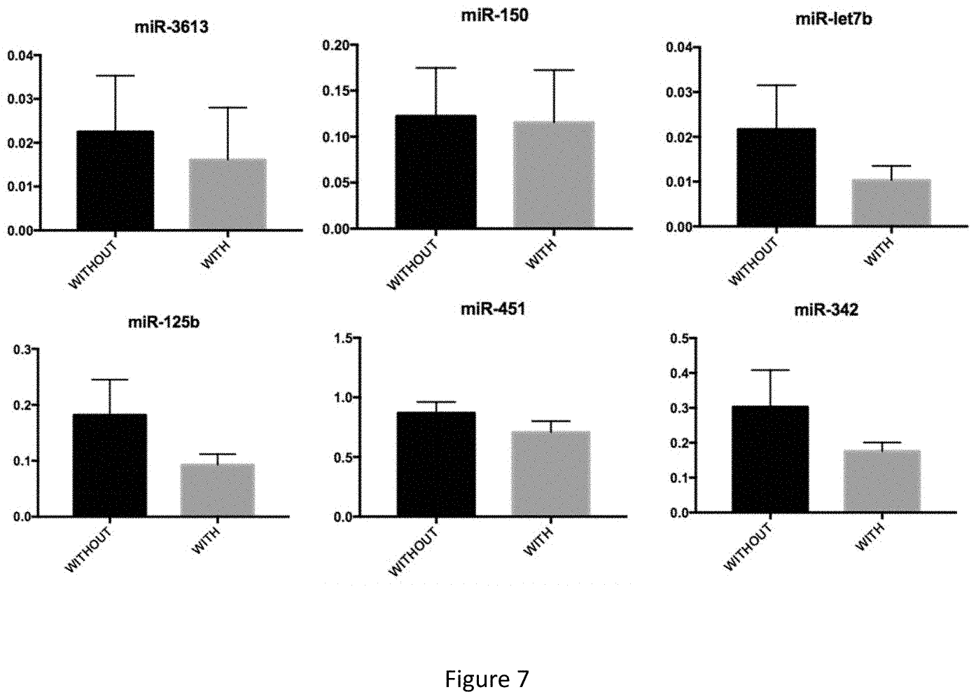

Quantitative PCR (Qt-PCR) was used to compare expression of microRNAs between endometriosis patients who received hormonal therapy and those that did not (FIG. 7).

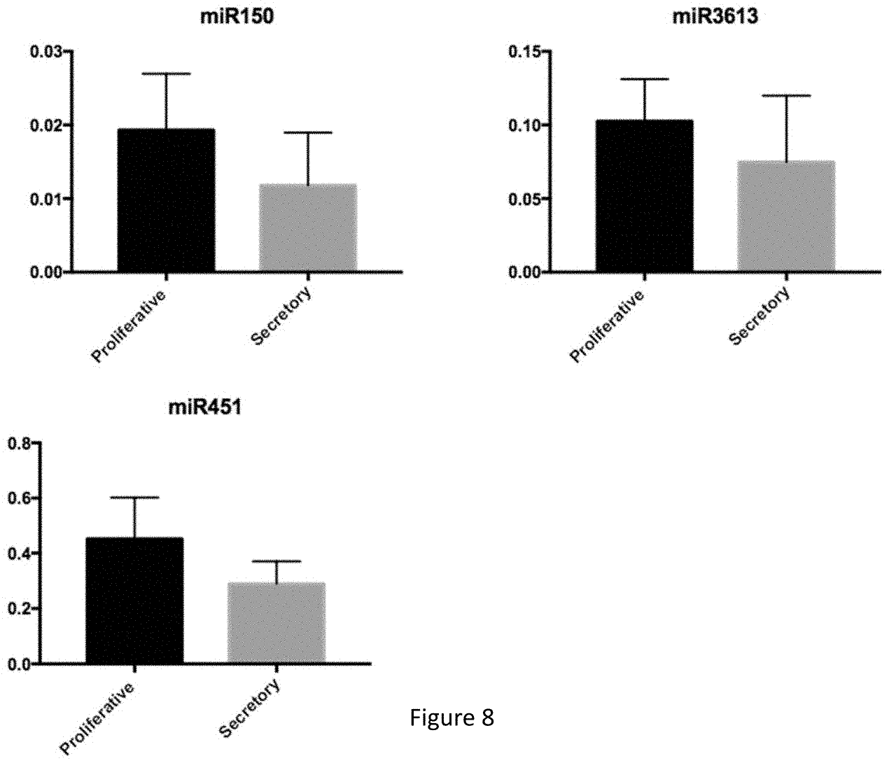

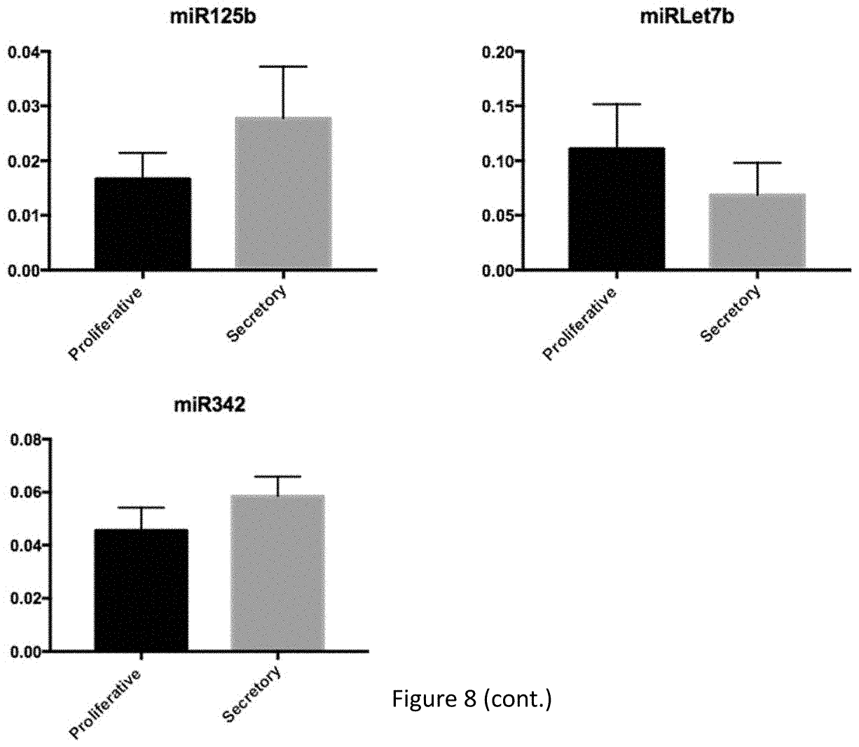

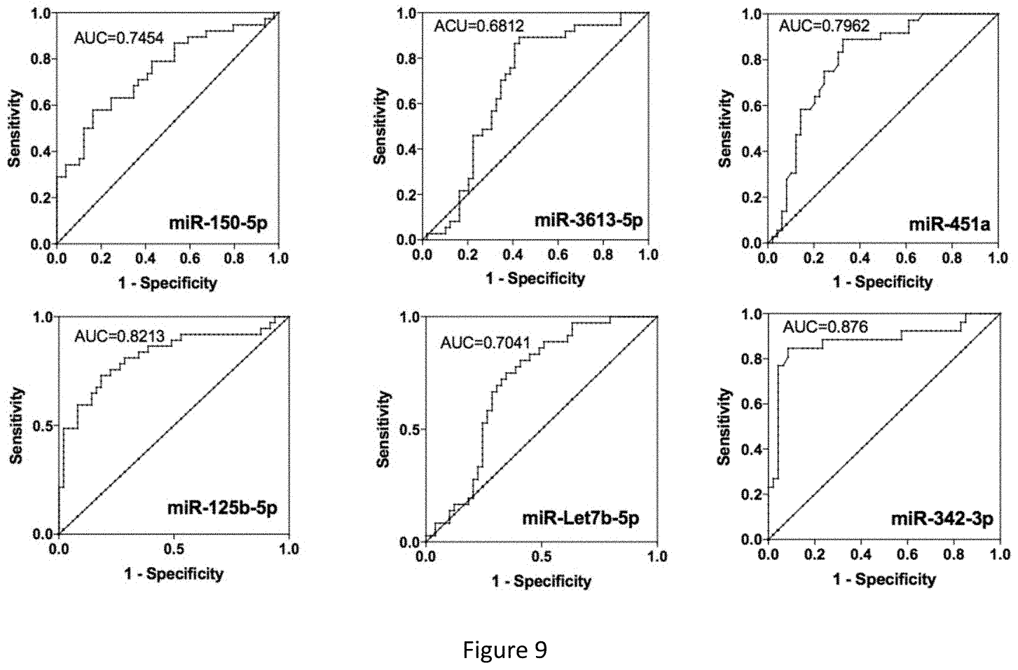

A subgroup analysis was also performed to assess whether timing of collection affected microRNA expression and accuracy of diagnosis with these markers. No specific difference in expression was noted between proliferative and secretory phase (FIG. 8). Receiver operating characteristic (ROC) curves and the areas under the ROC curve (AUC) were established to evaluate the diagnostic value of individual plasma microRNAs for differentiating between endometriosis and control groups (FIG. 9). miR-125 and miR-342 showed the greatest individual diagnostic value.

In addition to evaluating each identified biomarker individually, groups of miRNAs were evaluated. The combination of miR-125, miR-451, and miR-3613 yielded high diagnostic value, with an AUC of 0.917. in this study population. An alternative combination using let7b-, miR-150, miR-342, and miR-451 also yielded high diagnostic value, with an AUC of 0.977 in this study population.

This study identified miRNAs can reliably be used to differentiate between endometriosis and other gynecologic pathologies. In patients with endometriosis there is significantly lower expression of serum miRNAs miR-3613 and let7b- and significantly higher expression of serum miRNAs miR-150, miR-125b, miR-451, and miR-342. Further this study demonstrates that the combination of let-7b, miR-150, miR-342, miR-451 or the combination of miR-125, miR-451, and miR-3613 can be used to diagnose endometriosis with high sensitivity and specificity.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of assessing endometriosis, comprising:
a. inputting a level of at least one miRNA into an algorithm, wherein the at least one miRNA is selected from the group consisting of miR-18, miR-18a, miR-18a-5p, miR-34c, miR-125, miR-125b, miR-125b-5p, miR-126, miR-135, miR-141, miR-145, miR-143, miR-143-3p, miR-145-5p, miR-150, miR-150-5p, miR-200, miR-200a, miR-200b, miR-214, miR-342, miR-342-3p, miR-449, miR-449a, miR-451, miR-451a, miR-500, miR-500a, miR-500a-3p, miR-553, miR-3613, miR-3613-5p, miR-4668, miR-6755, miR-6755-3p, and let-7b,
b. quantitatively assessing the performance of the at least one miRNA or the algorithm using receiver operating characteristic (ROC) analysis and understanding AUC (Area Under the Curve),
c. combining at least one of the at least one miRNA into a mathematical formula,
d. assigning weights to at least one of the at least one miRNA within the formula, and
e. using the assigned weights to develop a quantitative algorithm to distinguish the presence or absence of disease.

2. The method of claim 1 further comprising one or more of:
f developing a quantitative algorithm to determine the extent, severity, or stage of disease,
g. developing a quantitative algorithm to determine a treatment approach (e.g., oral contraceptives, disease-specific therapy, surgical intervention),
h. developing a quantitative algorithm to select the appropriate dose for a medical treatment,
i. developing a quantitative algorithm to determine whether a patient is likely to respond to a particular medical or surgical treatment,
j. developing a quantitative algorithm to monitor response to treatment, and
k. developing a quantitative algorithm to monitor disease progression.

3. The method of any of claims 1-2 further comprising deriving a numerical value or score from the quantitative algorithm or mathematical formula using at least one miRNA.

4. The method of claim 3 further comprising establishing cutoff values for the derived numerical value or score from the quantitative algorithm for endometriosis.

5. The method of claim 4, comprising at least one of:
a. establishing cutoff values for at least one miRNA,
b. establishing cutoff values for the derived numerical value or score from one or more miRNAs in order distinguish the presence or absence of disease,
c. establishing cutoff values for the derived numerical value or score from one or more miRNAs in order determine the extent, severity, or stage of disease,
d. establishing cutoff values for the derived numerical value or score from one or more miRNAs in order to determine the right treatment approach (e.g., oral contraceptives, disease-specific therapy, surgical intervention),
e. establishing cutoff values for the derived numerical value or score from one or more miRNAs in order to select the appropriate dose for a medical treatment,
f. establishing cutoff values for the derived numerical value or score from one or more miRNAs in order to determine whether a patient is likely to respond to a particular medical or surgical treatment,
g. establishing cutoff values for the derived numerical value or score from one or more miRNAs in order to monitor response to treatment, and
h. establishing cutoff values for the derived numerical value or score from one or more miRNAs in order to monitor disease progression.

6. The method of any of claims 1-5 further comprising obtaining results derived from a quantitative algorithm for endometriosis.

7. The method of claim 5, wherein the quantitative algorithm is for determining the treatment approach (e.g., oral contraceptives, disease-specific therapy, surgical intervention).

8. The method of claim 5, wherein the quantitative algorithm is for selecting the appropriate dose for a medical treatment.

9. The method of claim 5, wherein the quantitative algorithm is for determining whether a patient is likely to respond to a particular medical or surgical treatment.

10. The method of claim 5, wherein the quantitative algorithm is used for monitoring response to treatment.

11. The method of claim 5, wherein the quantitative algorithm is used for monitoring disease progression.

12. The method of claim 1, wherein the at least one miRNA is selected from the group consisting of miR-135, miR-449a, miR-34c, miR-200a, miR-200b, miR-141, miR-125b-5p, miR-150-5p, miR-143-3p, miR-500a-3p, miR-18a-5p, miR-6755-3p, and miR-3613-5p.

13. The method of claim 1, wherein the at least one miRNA comprises:
(i) miR-150, miR-342, miR-451, and let-7b;
(ii) miR-125, miR-451, and miR-3613;
(iii) miR-125, miR-150, miR-342, and miR-451;
(iv) miR-125, miR-150, miR-342, miR-451, let-7b, and miR-3613; or
(v) miR-125 and miR-342.

14. The method of claim 1, wherein the quantitative algorithm is a fisher discriminant algorithm or a support vector machine algorithm.

15. A method of diagnosing a subject suspected of having endometriosis comprising:
a. obtaining a blood, serum, plasma, saliva, sputum, urine, lymphatic fluid, synovial fluid, cerebrospinal fluid, stool, or mucus sample from the subject, wherein the blood, serum, plasma, saliva, sputum, urine, lymphatic fluid, synovial fluid, cerebrospinal fluid, stool, or mucus sample comprises miRNA associated with endometriosis;
b. detecting a level of at least one miRNA selected from the group consisting of miR-125, miR-150, miR-342, miR-145, miR-143, miR-500, miR-451, miR-18, miR-214, miR-126, miR-6755, miR-3613, miR-553, miR-4668, let-7b, miR-135, miR-449a, miR-34c, miR-200a, miR-200b, miR-141, miR-125b-5p, miR-150-5p, miR-342-3p, miR-145-5p, miR-143-3p, miR-500a-3p, miR-451a, miR-18a-5p, miR-6755-3p, and miR-3613-5p;
c. comparing the level of the at least one miRNA with a cutoff value for the at least one miRNA derived from a quantitative algorithm for endometriosis; and
d. diagnosing the subject with endometriosis based on the comparison of the level of the at least one miRNA to the cutoff value.

16. The method of claim 15, wherein the at least one miRNA is the combination of miR-150, miR-342, miR-451 and let-7b.

17. The method of claim 16, wherein the at least one miRNA is the combination of an increased level of miR-150, an increased level of miR-342, an increased level of miR-451 and a decreased level of let-7b relative to the level in a comparator control.

18. The method of claim 17, wherein the increased level of miR-150 is at least 9-fold increased relative to the level in the comparator control; the increased level of miR-451 is at least 2-fold increased relative to the level in the comparator control; the decreased level of let-7b is at least 8-fold decreased relative to the level in the comparator control, or any combination thereof.

19. The method of claim 17 or 18, wherein the comparator control is the level of the miRNA in a population without endometriosis.

20. The method of claim 15, wherein the at least one miRNA is the combination of miR-125, miR-451, and miR-3613.

21. The method of claim 20, wherein the at least one miRNA is the combination of an increased level of miR-125, an increased level of miR-451, and a decreased level of miR-3613 relative to the level in a comparator control.

22. The method of claim 21, wherein the increased level of miR-125 is at least five-fold increased relative to the level in the comparator control; the increased level of miR-451 is at least 2-fold increased relative to the level in the comparator control; the decreased level of miR-3613 is at least four-fold decreased relative to the level in the comparator control, or any combination thereof.

23. The method of claim 21 or 22, wherein the comparator control is the level of the miRNA in a population without endometriosis.

24. The method of claim 15, further comprising administering a treatment to the subject for endometriosis.

25. The method of claim 24, wherein the treatment is at least one treatment selected from the group consisting of hormone therapy, chemotherapy, immunotherapy, and surgical treatment.

26. A method of detecting endometriosis using a quantitative polymerase chain reaction (qPCR) machine or sequencing machine comprising:
a. introducing nucleic acids into the qPCR machine or sequencing machine, wherein nucleic acids are derived from a sample obtained from a female subject with endometriosis or with symptoms of endometriosis;
b. using the qPCR machine or sequencing machine to detect a level of at least one miRNA in the nucleic acids, wherein the at least one miRNA is selected from the group consisting of miR-18, miR-18a, miR-18a-5p, miR-34c, miR-125, miR-125b, miR-125b-5p, miR-126, miR-135, miR-141, miR-145, miR-143, miR-143-3p, miR-145-5p, miR-150, miR-150-5p, miR-200, miR-200a, miR-200b, miR-214, miR-342, miR-342-3p, miR-449, miR-449a, miR-451, miR-451a, miR-500, miR-500a, miR-500a-3p, miR-553, miR-3613, miR-3613-5p, miR-4668, miR-6755, miR-6755-3p, and let-7b, thereby obtaining a detected level of the at least one miRNA;
c. introducing the detected level of the at least one miRNA into a trained algorithm wherein the algorithm:
(i) is trained using data from endometriosis subjects;
(ii) assigns weights to the at least one miRNA selected from the group consisting of miR-18, miR-18a, miR-18a-5p, miR-34c, miR-125, miR-125b, miR-125b-5p, miR-126, miR-135, miR-141, miR-145, miR-143, miR-143-3p, miR-145-5p, miR-150, miR-150-5p, miR-200, miR-200a, miR-200b, miR-214, miR-342, miR-342-3p, miR-449, miR-449a, miR-451, miR-451a, miR-500, miR-500a, miR-500a-3p, miR-553, miR-3613, miR-3613-5p, miR-4668, miR-6755, miR-6755-3p, and let-7b; and
(iii) detects endometriosis based on the assigned weights of the at least one miRNA; and
(d) using the trained algorithm to detect presence of absence of endometriosis in the female subject.

27. The method of claim 26, wherein the at least one miRNA is selected from the group consisting of miR-135, miR-449a, miR-34c, miR-200a, miR-200b, miR-141, miR-125b-5p, miR-150-5p, miR-143-3p, miR-500a-3p, miR-18a-5p, miR-6755-3p, and miR-3613-5p.

28. The method of claim 26, wherein the at least one miRNA comprises:
(i) miR-150, miR-342, miR-451, and let-7b;
(ii) miR-125, miR-451, and miR-3613;
(iv) miR-125, miR-150, miR-342, and miR-451;
(iv) miR-125, miR-150, miR-342, miR-451, let-7b, and miR-3613; or
(v) miR-125 and miR-342.

29. The method of claim 26, 27, or 28, wherein the trained algorithm is a support vector machine algorithm or fisher discriminant algorithm;

30. The method of claim 26, wherein the method uses a qPCR machine.

31. The method of claim 26, wherein the method uses a sequencing machine.

32. The method of claim 31, wherein the sequencing machine is a next-generation sequencing machine.

33. The method of claim 26, comprising using the trained algorithm to detect the presence of endometriosis in the female subject.

34. The method of claim 33, further comprising administering a treatment to the female subject for the endometriosis.

35. The method of claim 34, wherein the treatment is selected from the group consisting of: hormone therapy, chemotherapy, immunotherapy, and surgical treatment.

36. The method of any one of the preceding claims, wherein the sample is a blood, plasma or serum sample.

37. The method of any one of the preceding claims, wherein the sample is a saliva or sputum sample.

38. The method of claim 26, wherein the at least one miRNA is the combination of miR-150, miR-342, miR-451 and let-7b.

39. The method of claim 38, wherein the endometriosis is detected when an increased level of miR-150, an increased level of miR-342, an increased level of miR-451 and a decreased level of let-7b relative to the level in a comparator control is detected.

40. The method of claim 39, wherein the increased level of miR-150 is at least 9-fold increased relative to the level in the comparator control; the increased level of miR-451 is at least 2-fold increased relative to the level in the comparator control; the decreased level of let-7b is at least 8-fold decreased relative to the level in the comparator control, or any combination thereof.

41. The method of claim 39 or 40, wherein the comparator control is the level of the miRNA in a population without endometriosis.

42. The method of claim 26, wherein the at least one miRNA is the combination of miR-125, miR-451, and miR-3613.

43. The method of claim 42, wherein the endometriosis is detected when an increased level of miR-125, an increased level of miR-451, and a decreased level of miR-3613 relative to the level in a comparator control is detected.

44. The method of claim 43, wherein the increased level of miR-125 is at least five-fold increased relative to the level in the comparator control; the increased level of miR-451 is at least 2-fold increased relative to the level in the comparator control; the decreased level of miR-3613 is at least four-fold decreased relative to the level in the comparator control, or any combination thereof.

45. The method of claim 43 or 44, wherein the comparator control is the level of the miRNA in a population without endometriosis.

46. The method of claim 35, wherein the treatment is selected from the group consisting of oral contraceptive pill, GNRH agonists, aromatase inhibitors, and progesterone.
